(19) United States
(12) Patent Application Publication
(10) Pub. No.: US 2021/0330212 A1
(43) Pub. Date: Oct. 28, 2021

(54) ERROR CORRECTION TECHNIQUES ON BIO-IMPEDANCE MEASUREMENTS

(71) Applicant: Analog Devices International Unlimited Company, Limerick (IE)

(72) Inventors: Sriram GANESAN, Bangalore (IN); Venugopal GOPINATHAN, Boston, MA (US); Tony J. AKL, Bedford, MA (US)

(73) Assignee: Analog Devices International Unlimited Company, Limerick (IE)

(21) Appl. No.: 17/370,173

(22) Filed: Jul. 8, 2021

Related U.S. Application Data
(63) Continuation of application No. PCT/EP2020/050545, filed on Jan. 10, 2020.
(60) Provisional application No. 62/790,619, filed on Jan. 10, 2019.

(51) Int. Cl. A61B 5/053; A61B 5/00

(52) U.S. Cl. CPC A61B 5/053; A61B 5/7225

(57) ABSTRACT

Determining bio-impedance of a body, or portion thereof, of a subject has been utilized for determining health characteristics (such as heart conditions) of the subject. The systems and procedures described herein may provide for correction and/or compensation for electrode contact impedance and for accurately determining bio-impedance. The system may take into account impedance sensitivity and/or frequency sensitivity when performing the bio-impedance determination to improve the bio-impedance determination.

### RELATED APPLICATIONS

The present disclosure claims priority to, as a bypass continuation, International Patent Application Serial No. PCT/EP2020/050545, entitled “ERROR CORRECTION TECHNIQUES ON BIO-IMPEDANCE MEASUREMENTS” and filed on Jan. 10, 2020. The International Patent Application claims priority to and receives benefit of U.S. Provisional Application No. 62/790,619 entitled “ERROR CORRECTION TECHNIQUES ON BIO-IMPEDANCE MEASUREMENTS” and filed Jan. 10, 2019, the disclosure of which is incorporated by reference in its entirety.

### FIELD OF THE DISCLOSURE

This disclosure relates in general to the field of impedance measurements, and more particularly, though not exclusively, to a system and method for compensating for errors due to electrode contact impedance.

### BACKGROUND

Impedance measurements of the body, referred to herein as bio-impedance, has many applications in healthcare and consumer applications. Impedance measurements can be made by electrodes provided in body-worn systems, or wearable devices, such as wrist watches, chest bands, head bands, patches, and so on. Circuitry coupled to the electrodes can derive the unknown impedance of the body on which the electrodes are placed. Impedance measurements can be particularly useful for vital-signs monitoring, sensing of tissues and fluid level in the body for purposes of detecting signs of congestive heart failure, electrical impedance tomography systems, electrical impedance spectroscopy, etc.

### BRIEF DESCRIPTION OF THE DRAWINGS

The present disclosure is best understood from the following detailed description when read with the accompanying figures. It is emphasized that, in accordance with the standard practice in the industry, various features are not necessarily drawn to scale, and are used for illustration purposes only. Where a scale is shown, explicitly or implicitly, it provides only one illustrative example. In other embodiments, the dimensions of the various features may be arbitrarily increased or reduced for clarity of discussion.

FIG. 1 illustrates an example system having electrodes and circuitry for performing a four-way impedance measurement of bio-impedance, according to some embodiments of the disclosure.

FIG. 2 illustrates example input capacitances that can be present in circuitry that can perform a four-way impedance measurement of bio-impedance, according to some embodiments of the disclosure.

FIG. 3 illustrates example current leakage present in circuitry that performs a four-way impedance measurement of bio-impedance, according to some embodiments of the disclosure.

FIG. 4 illustrates circuitry which can perform a measurement that is less sensitive to errors caused by impedances on the S+ branch and the S− branch, according to some embodiments of the disclosure.

FIG. 5 is a flow diagram illustrating a method for performing a measurement that is less sensitive to errors caused by impedances on the S+ and S− branches, according to some embodiments of the disclosure.

FIG. 6 is a flow diagram illustrating a method for performing a measurement that is less sensitive to errors associated with frequency, according to some embodiments of the disclosure.

FIG. 7 illustrates an example measurement arrangement that can implement the techniques described herein, according to some embodiments of the disclosure.

FIG. 8 illustrates an example system arrangement that can implement the techniques described herein, according to some embodiments of the disclosure.

### SUMMARY OF THE DISCLOSURE

Systems and procedures described herein may provide for corrected and/or compensated voltage measurements of a bio-impedance of a body of a subject. For example, systems for measuring bio-impedance (such as four-wire systems described herein) may have sources of error in measuring the bio-impedance that can result in the measurement of the bio-impedance being inexact. Techniques related to impedance sensitivity and frequency sensitivity disclosed herein may be implemented to correct and/or compensate for voltage measurement of the bio-impedance that can improve the determination of the value of the bio-impedance.

Some embodiments may include circuitry for determining an amount of a bio-impedance of a portion of a body of a subject. The circuitry may comprise first impedance circuitry coupled to a first pin of the circuitry, the first pin to be coupled to a first side of the portion of the body, wherein the first impedance circuitry is to selectively couple a first impedance to the first pin. The circuitry may further comprise second impedance circuitry coupled to a second pin of the circuitry, the second pin to be coupled to a second side of the portion of the body, wherein the second impedance circuitry is to selectively couple a second impedance to the second pin. The circuitry may further comprise voltage measurement circuitry coupled to the first pin and the second pin, the voltage measurement circuitry to determine a first voltage difference between the first pin and the second pin with the first impedance coupled to the first pin and the second impedance decoupled from the second pin, and determine a second voltage difference between the first pin and the second pin with the first impedance decoupled from the first pin and the second impedance coupled to the second pin, the first voltage difference and the second voltage difference to be utilized for compensation to determine the amount of the bio-impedance.

Some embodiments may include a system for determining a value of a bio-impedance of a portion of a body of a subject. The system may comprise a first electrode to be positioned on a first end of the portion of the body and a second electrode to be positioned on a second end of the portion of the body. The system may further include circuitry coupled to the first electrode and the second electrode, the circuitry to determine voltage differences between the first electrode and the second electrode. The circuitry may comprise first impedance circuitry coupled to the first electrode, the first impedance circuitry to selectively couple a first impedance between the first electrode and a ground of the circuitry. The circuitry may further comprise second impedance circuitry coupled to the second electrode, the second impedance circuitry to selectively couple a second impedance between the second electrode and the ground of the circuitry. The circuitry may further comprise voltage measurement circuitry coupled to the first electrode and the second electrode, the voltage measurement circuitry to determine the voltage differences between the first electrode and the second electrode with selective coupling of the first impedance between the first electrode and the ground of the circuitry and selective coupling of the second impedance between the second electrode and the ground of the circuitry.

Some embodiments may include a process for determining a value of a bio-impedance of a portion of a body of a subject. The process may comprise determining, by circuitry, a first voltage difference between a first electrode positioned at a first end of the portion of the body and a second electrode positioned at a second end of the portion of the body with the circuitry having a first configuration, changing, by the circuitry, from the first configuration to a second configuration after the first voltage difference is determined, and determining, by the circuitry, a second voltage difference between the first electrode and the second electrode with the circuitry having the second configuration, the first voltage difference and the second voltage difference to be utilized for compensation to determine the value of the bio-impedance.

### DETAILED DESCRIPTION

The following disclosure provides many different embodiments, or examples, for implementing different features of the present disclosure. Specific examples of components and arrangements are described below to simplify the present disclosure. These are, of course, merely examples and are not intended to be limiting. Further, the present disclosure may repeat reference numerals and/or letters in the various examples, or in some cases across different figures. This repetition is for the purpose of simplicity and clarity and does not in itself dictate a specific relationship between the various embodiments and/or configurations discussed. Different embodiments may have different advantages, and no particular advantage is necessarily required of any embodiment.

Bio-impedance measurements of a body, or portions thereof, of a subject can be utilized for determining health characteristics (such as heart conditions) of the subject. However, performing bio-impedance measurements may present sources of error that result in the bio-impedance measurement being imprecise. For four-way or four-wire impedance measurement approaches of measuring the bio-impedance, the measurement system may present parasitic capacitances that can cause the measured bio-impedance to be imprecise.

The systems and procedures described herein may take into account impedance sensitivity and/or frequency sensitivity that can be utilized for correcting and/or compensating measurements of bio-impedance. For example, the systems and procedures may include performing multiple measurements with different frequencies of signals applied to the bio-impedance and/or different impedances being coupled to the electrodes of the system utilized for measuring the bio-impedance to determine impedance sensitivity and/or frequency sensitivity of the bio-impedance measurement. The system and procedures may correct and/or compensate for the determined impedance sensitivity and/or frequency sensitivity.

It should be noted that throughout the FIGURES, certain reference numerals may be repeated to indicate that a particular device or block is wholly or substantially consistent across the FIGURES. This is not, however, intended to imply any particular relationship between the various embodiments disclosed. In certain examples, a genus of elements may be referred to by a particular reference numeral (“widget 10”), while individual species or examples of the genus may be referred to by a hyphenated numeral (“first specific widget 10-1” and “second specific widget 10-2”).

Four-Way or Four-Wire Impedance Measurement

A technique for impedance measurement of a bio-impedance may comprise a four-terminal sensing scheme, which may also be referred to as a four-way impedance measurement scheme, four-wire sensing, or Kelvin sensing. The bio-impedance may be produced by a body, or portion thereof, of a subject. In particular, the bio-impedance may comprise an impedance presented by the body, or portion thereof, of the subject to the flow of current that may be applied to the body. The technique may involve having a plurality of electrodes placed on a body of a subject. For example, the technique may involve having four electrodes placed on a body of a subject, and the electrodes may be utilized to sense or derive an unknown bio-impedance of a portion of the body of the subject corresponding to the placement of the electrodes. In some instances, the bio-impedance of the portion of the body may measure is on the order of 100 Ohms.

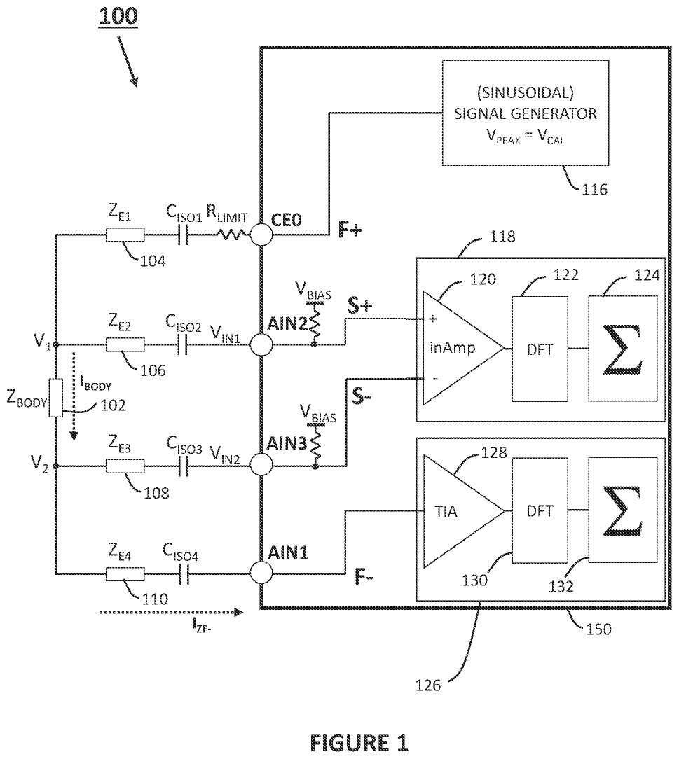

FIG. 1 illustrates an example system 100 having electrodes and circuitry for performing a four-way impedance measurement of bio-impedance, according to some embodiments of the disclosure. In the illustrated embodiment, the unknown bio-impedance 102 is shown as Z_{BODY}. The bio-impedance 102 may represent a bio-impedance produced by a body, or portion thereof, of a subject. As used throughout this disclosure, relationships of elements to the bio-impedance 102 may refer to relationships of the elements to the body of the subject. For example, references to voltage being applied across the bio-impedance 102 may refer to voltage being applied to the body, or portion thereof, that produces the bio-impedance 102. Further, references to electrodes being coupled to a first end of the bio-impedance 102 and the second end of the bio-impedance 102 may refer to the electrodes being coupled to a first end of the body, or portion thereof, and a second end of the body, or portion thereof, that produces the bio-impedance 102.

The system 100 may include a plurality of electrodes. In the illustrated embodiment, the system 100 includes four electrodes: electrode 104, electrode 106, electrode 108, and electrode 110. The electrodes may contact a body of a subject. For example, the electrodes may be positioned on the body of the subject, where the electrode may be positioned in different positions on the body of the subject.

Each of the electrodes may present a respective impedance based on a contact of the electrodes with the body of the subject. The amount of impedance presented by each of the electrodes may depend on a quality of contact with the body of the subject. The electrode 104 presents a respective contact impedance Z_{E1}, the electrode 106 presents a respective contact impedance Z_{E2}, the electrode 108 presents a respective contact impedance Z_{E3}, and the electrode 110 present a respective contact impedance ZE₄.

The system may further include circuitry 150 coupled to the electrodes. In some embodiments, the circuitry 150 may be packaged as an integrated circuit or chip. The circuitry 150 may include pins (or connectors) to which the electrodes may be coupled. For example, the circuitry 150 may have a pin CE0 that is electrically coupled to electrode 104. The circuitry 150 may further include a pin AIN2 that is electrically coupled to electrode 106. The circuitry 150 may further include a pin AIN3 that is electrically coupled to electrode 108. The circuitry 150 may further include a pin AIN1 that is electrically coupled to electrode 110.

The system 100 may include a plurality of branches, where each branch may include an electrode and a pin. For example, the system 100 has four branches in the illustrated embodiment: an F+ branch that includes the electrode 104 and the pin CE0, an S+ branch that includes the electrode 106 and the pin AIN2, an S− branch that includes the electrode 108 and the pin AIN3, and an F− branch that includes the electrode 110 and the pin AIN1. The F+ branch, that includes the electrode 104, may be coupled to the first end of a portion of the body of a subject that produces the unknown bio-impedance 102. The S+ branch, that includes the electrode 106, may be coupled to the first end of the portion of the body of the subject that produces the unknown bio-impedance 102. The S− branch, that includes the electrode 108, may be coupled to the second end of the portion of the body of the subject that produces the unknown bio-impedance 102. The F− branch, that includes electrode 110, may be coupled to the second end of the portion of the body of the subject that produces the unknown bio-impedance 102. The four branches are connected to respective pins of circuitry 150. The F+ branch and the F− branch can be referred to as the force lines. The S+ branch and the S− branch can be referred to as the sense lines. Portions of each of the branches illustrated outside of circuitry 150 can represent cables that extend from the circuitry 150 and the electrodes at an opposite end of the cables from the circuitry 150, where the electrodes may be implemented in patches at the opposite end of the cables. In other embodiments, the portions of each of the branches illustrated outside of circuitry 150 can represent conductors or wires having electrodes at the end of the conductors or wires. The conductors and electrodes can be fitted in a wearable device in some embodiments. Optionally, isolation circuitry may be included between the electrodes and the pins to provide isolation and protection between the body of the subject and the circuitry 150. The isolation circuitry may include and/or produce capacitances C_{ISO1}, C_{ISO2}, C_{ISO3}, C_{ISO4} positioned between the respective electrodes and pins to provide isolation and protection between the body of the human user and the circuitry 150. For example, the capacitance C_{ISO1}, the capacitance C_{ISO2}, the capacitance C_{ISO3}, and the capacitance C_{ISO4} can be included between respective pairs of electrodes and pins to provide isolation and protection between the body of the human user and the circuitry within circuitry 150 (e.g., to block DC signals). In instances where the isolation circuitry includes the isolation circuitry, the capacitances C_{ISO1}, C_{ISO2}, C_{ISO3}, C_{ISO4} may be included in the respective branches.

Circuitry 150 can include a signal generator 116 (e.g., sinusoidal signal generator). The signal generator 116 can generate a signal having a peak voltage of VPEAK. The signal generator 116 can generate various kinds of signals. For instance, the signal generator 116 can generate signals at different frequencies. For purposes of measuring bio-impedance, signals having different frequencies from 0 Hz to hundreds/thousands/millions of Hz can be used. Applications, such as impedance tomography and impedance spectroscopy, often benefit from using signals having wide range of frequencies. The signal generator 116 can apply signals to the body, or portion thereof, that produce the bio-impedance 102, which may cause current to flow across the bio-impedance 102 and a voltage drop to be produced across the unknown bio-impedance 102. The signal generator 116, in some implementations, can force a current to flow through the unknown bio-impedance 102.

The circuitry 150 can include voltage measurement circuitry 118 to measure a voltage between a positive input and a negative input of the voltage measurement circuitry 118. In the example shown, the positive input is coupled to the S+ branch, and the negative input is coupled to the S− branch to measure a voltage difference between V_{IN1} and V_{IN2}, e.g., V_{IN1}−V_{IN2}. For example, the positive input of the voltage measurement circuitry 118 may be coupled to the pin AIN2 and the negative input of the voltage measurement circuitry 118 may be coupled to the pin AIN3. In some embodiments, the voltage measurement circuitry 118 can include an instrumentation amplifier (inAmp) 120 with a positive terminal and a negative terminal to sense a voltage difference between the positive terminal and negative terminal, and outputs a voltage output representative of the voltage difference. In some embodiments, the positive terminal of the inAmp 120 may be coupled to the pin AIN2 and the negative terminal of the inAmp 120 may be coupled to the pin AIN3. The voltage measurement circuitry 118 can include a Discrete Fourier Transform (DFT) block 122 and summation block 124 to generate a voltage measurement based on the voltage output from inAmp 120.

The circuitry 150 can further include current measurement circuitry 126 to measure a current at an input of the current measurement circuitry 126. In the example shown, the input is coupled to the F− branch to measure the current flowing through the F− branch. In some embodiments, current measurement circuitry 126 can include a transimpedance amplifier (TIA) 128 to convert a current at an input terminal of the TIA 128 to a voltage output representative of the current. The current measurement circuitry 126 can include a DFT block 130 and a summation block 132 to generate a current measurement based on the voltage output from the TIA 128.

To make an impedance measurement, a voltage drop may be generated across the unknown bio-impedance 102. The voltage drop across the unknown bio-impedance 102 can be viewed as V₁−V₂. The voltage drop across the unknown bio-impedance 102 can be generated or imposed by the signal generator 116. For example, the signal generator 116 may apply a signal to the body of the subject via the electrode 104, where the signal causes the voltage drop to be generated across the bio-impedance 102 based on a current flow across the bio-impedance and a value of the bio-impedance 102. Meanwhile, the voltage drop across the unknown bio-impedance 102, may be measured by the voltage measurement circuitry 118, and a current through the unknown bio-impedance 102 can also be measured by current measurement circuitry 126. The measured voltage drop and the measured current can be used to derive the impedance value of the unknown bio-impedance 102. The circuitry 150 can include circuitry and/or one or more processors (not shown) to derive the impedance value of the unknown bio-impedance 102, based on the measured voltage from the voltage measurement circuitry 118 and measured current from the current measurement circuitry 126. In other embodiments, the circuitry 150 may include communication circuitry to wiredly or wirelessly communicate the measured voltage and/or the measured current to a remote device (such as a server, computer, or other computing device located remote to the circuitry 150), where the remote device may derived the value of the impedance of the bio-impedance 102 based on the measured voltage and/or the measured current.

The measurement scheme assumes, in an ideal situation, that the input impedance of the inAmp 120 is infinite, and no current flows through the S+ branch and S− branch. In this ideal situation, all of the current I_{BODY} that flows through the unknown bio-impedance 102 would flow through the F− branch, meaning I_{BODY}=I_{ZF-}. When no current is drawn through impedance Z_{E2} and impedance Z_{E3}, V_{IN1}=V₁, and V_{IN2}=V₂, and therefore, the voltage difference measured by voltage measurement circuitry 118 represents the voltage across the unknown bio-impedance 102, i.e., V_{IN1}−V_{IN2}=V₁−V₂.

In legacy two-way impedance measurements, measurement issues can arise from impedances of cables (including the contact impedances of the electrodes) being added to the unknown bio-impedance 102, thus corrupting the impedance measurement. For simplicity, the impedances present are lumped together as the illustrated contact impedances in each of the branches. In theory, a four-way impedance measurement can avoid such issues. Moreover, when the unknown bio-impedance 102 is much higher than the impedances of the cables, the measurements can be sufficiently accurate. However, the setup seen in FIG. 1, in practice, can have certain other limitations or non-idealities. These limitations can be significant, e.g., when making impedance measurements at low frequencies, high frequencies, certain frequencies, or various frequencies. In some situations, sometimes one or more of the contact impedance Z_{E1}, contact impedance Z_{E2}, contact impedance Z_{E3}, and contact impedance Z_{E4} can be greater than the unknown bio-impedance 102. For instance, mechanical and/or environmental reasons (e.g., humidity, movement, etc.) can cause poor contacts, and can severely increase one or more of the contact impedances. For instance, the (magnitude of) contact impedances can be greater than 2 kΩ. In some situations, the optional capacitor C_{ISO1}, the optional capacitor C_{ISO2}, the optional capacitor C_{ISO3}, and the optional capacitor C_{ISO4} for isolation can also significantly increase or affect the impedances of the cables. In some situations, the contact impedance Z_{E1}, the contact impedance Z_{E2}, the contact impedance Z_{E3}, and the contact impedance Z_{E4} can have an imbalance with each other (e.g., imbalance can be greater than 1 KΩ). These limitations have been found to degrade the accuracy of the four-way impedance measurement.

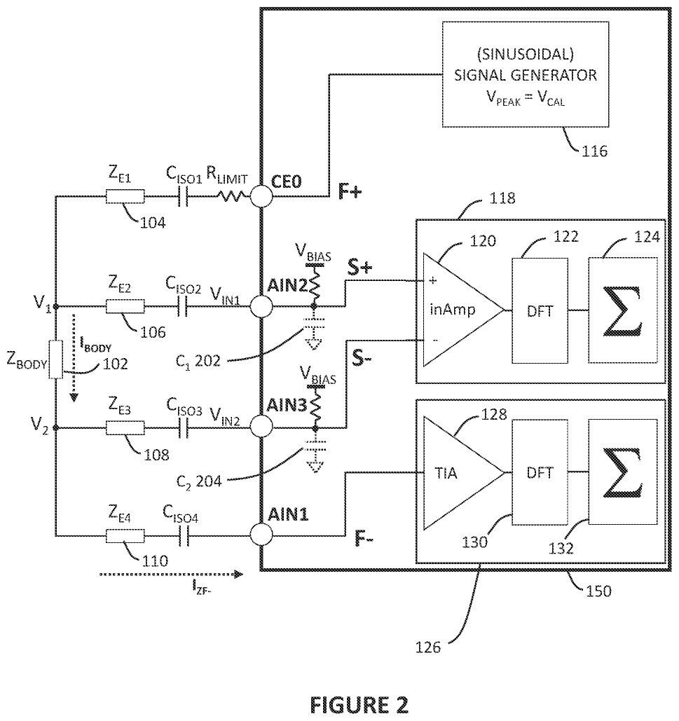

One aspect that can cause these limitations to degrade the accuracy of the bio-impedance measurement is that there can be large input capacitances at pin AIN2 and pin AIN3 (e.g., around 40 picofarads (pF)). FIG. 2 illustrates example input capacitances (e.g., parasitic capacitances in the printed circuit board, printed circuit board track capacitance, and/or wiring/conductor capacitances) that can be present in circuitry that can perform a four-way impedance measurement of bio-impedance, according to some embodiments of the disclosure. Grounded input capacitance C₁ 202 can be present at pin AIN2, and grounded input capacitance C₂ 204 can also be present at pin AIN3. Grounded input capacitance C₁ 202 and capacitance C_{ISO2} can form a filter. Grounded input capacitance C₂ 204 and capacitance C_{ISO3} can also form a filter. Ideally, voltage V₁ should be the same as the voltage V_{IN1}, and voltage V₂ should be the same as the voltage V_{IN2}. Due to the grounded input capacitance C₁ 202 and grounded input capacitance C₂ 204, at certain frequencies, voltage V₁ may not be the same as the voltage V_{IN1}, and voltage V₂ may not be the same as the voltage V_{IN2}. The voltage across voltage V₁ and voltage V₂ may not be the same as the voltage across V_{IN1} and V_{IN2}. For example, in the case of bio-impedance measurements done at 100 kHz or higher frequencies, the input (parasitic) capacitance of the inAmp 120 lowers the input impedance of the inAmp 120 significantly. At 200 kHz, 10 pF of input (parasitic) capacitance is equivalent to 80 kOhm of resistance. The voltage drop across the contact impedances is no longer negligible, and the voltage measurement can be severely impacted. The negative effect of the grounded input capacitance C₁ 202 and the grounded input capacitance C₂ 204 can be observable at various frequencies and when contact impedances are high, e.g., in the range of hundreds or thousands of Ohms. Furthermore, the grounded input capacitance C₁ 202 and the grounded input capacitance C₂ 204 can attribute to imbalances in the contact impedances. Imbalances in the contact impedances of the branches can produce different cut-off frequencies, thereby causing different attenuations in each branch.

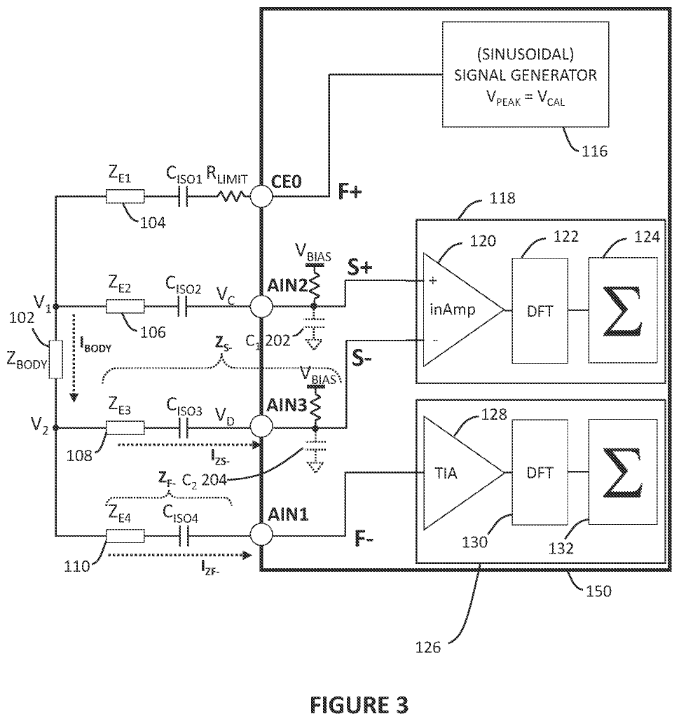

Another aspect that can cause these limitations to degrade the accuracy of the bio-impedance measurement is current leakage. FIG. 3 illustrates example current leakage present in circuitry that performs a four-way impedance measurement of bio-impedance, according to some embodiments of the disclosure. The current leakage can arise because an impedance Z_{S-} of the S− branch having electrode 108 can be similar to an impedance Z_{F-} of the F− branch having electrode 110 driving the TIA 128. This results in some of the current I_{BODY} that flows through the unknown bio-impedance 102 to flow through the S− branch having electrode 108, and not all of the current I_{BODY} would flow through the F− branch having electrode 110. In other words, the current I_{ZS-} through the S− branch having electrode 108 is ideally zero, and the current I_{ZF-} through the F− branch having electrode 110 is ideally equal to the current I_{BODY}. In reality, the current I_{ZS-} is not zero. As a result, the current I_{ZF-} through the branch having electrode 110 does not equal to current I_{BODY}, and part of the current I_{BODY} is not measured by the current measurement circuitry 126. Based on part of the current I_{BODY} not being measured by the current measurement circuitry 126, the current measurement would be corrupted, and thus the impedance measurement is also corrupted. This issue can be exacerbated by high contact impedances and/or imbalances of impedances in the branches.

As mentioned previously, in practice, some current can be leaked/drawn through the S+ branch and S− branch because the input impedance of the inAmp 120 is finite. The leakage of current through the S+ branch and S− branch can also corrupt the voltage measurement since an unknown voltage drop across the impedance Z_{E2} and/or an unknown voltage drop across the impedance Z_{E3} would mean that the voltage difference measured by the voltage measurement circuitry 118 no longer accurately represents the voltage across the unknown bio-impedance 102, i.e., V_{IN1}−V_{IN2}≠V₁−V₂. The measurement can be further corrupted by the presence of large contact impedances and/or imbalance in the impedances in the branches.

Approach to Addressing the Sources of Error

While the four-way impedance measurement scheme can be effective for measuring bio-impedance, techniques can be applied to correct or compensate for certain sources of error to make the four-way impedance measurement scheme more immune to the sources of error. Moreover, some reasonable assumptions can be made to allow the techniques to extend the tolerable contact impedance while still be able to measure the bio-impedance across a range of frequencies.

Specifically, two techniques (which can be used together or separately) for making the impedance measurements (more specifically, the voltage measurement performed by the inAmp 120) more immune to the sources of error that may be employed are described herein. One technique can expose the sensitivity of the measurement to impedance on the S+ branch and S− branch, and can be utilized to correct or compensate the measurement based on the impedance sensitivity. The other technique can expose the sensitivity of the measurement to frequency, and can be utilized to correct or compensate the measurement based on the frequency sensitivity.

Both techniques can involve making multiple measurements under different conditions, and can perform a linear combination of a measurement to be corrected or compensated and differences between the measurements to perform error correction or compensation. The resulting combination of measurements can advantageously take a sensitivity into account and perform an appropriate correction or compensation for the measurement.

Correction Based on Impedance Sensitivity

A first technique of the techniques for making the impedance more immune to the sources of errors may comprise correction based on impedance sensitivity. As discussed previously, unknown voltage drops across the impedance Z_{E2} and unknown voltage drops across the impedance Z_{E3} due to non-idealities, such as input (parasitic) capacitances and/or finite input impedances of the inAmp 120, can corrupt the four-way impedance measurement. Consider the case of input (parasitic) capacitances at the inAmp 120, it is possible to determine the error introduced by the input capacitances by computing the voltage difference measured by inAmp 120 in terms of the voltage across the unknown bio-impedance 102 (e.g., V₁−V₂) impedances on the S+ branch and S− branch and input capacitances of inAmp 120. The voltage V_{IN1} and the voltage V_{IN2} seen at the inputs of inAmp 120, in the presence of input (parasitic) capacitances C₁ and C₂, may be defined as follows:

$$\begin{matrix}{V}_{IN 1} = {V}_{1} - \frac{{V}_{1} {Z}_{1}}{{Z}_{1} + \frac{1}{j ω {C}_{1}}} & ( eq . 1 ) \\ {V}_{IN 2} = {V}_{2} - \frac{{V}_{2} {Z}_{2}}{{Z}_{2} + \frac{1}{j ω {C}_{2}}} & ( eq . 2 )\end{matrix}$$

The voltage V_{IN1} is the voltage at the positive input of inAmp 120. The voltage V_{IN2} is the voltage at the negative input of inAmp 120. The impedance Z₁ may include the contact impedance Z_{E2} of the S+ branch, or more broadly the impedance on the S+ branch including impedance resulting from the electrode 106 to the positive input of the inAmp 120. The impedance Z₂ may include the contact impedance Z_{E2} of the S− branch, or more broadly the impedance on the S+ branch including impedance resulting from the electrode 108 to the negative input of the inAmp 120. The capacitance C₁ (e.g., C₁ 202) represents the input (parasitic) capacitance at the positive input of the inAmp 120. The capacitance C₂ (e.g., C₂ 204) is the input (parasitic) capacitance at the negative input of the inAmp 120. The inAmp 120 measures a difference between voltage V_{IN1} and voltage V_{IN2} (i.e., V_{IN1}−V_{IN2}). A measured voltage difference that may be produced by the inAmp 120 can be defined as follows:

$${V}_{D 1} = {V}_{IN 1} - {V}_{IN 2} = {V}_{1} - \frac{{V}_{1} {Z}_{1}}{{Z}_{1} + \frac{1}{j ω {C}_{1}}} - {V}_{2} + \frac{{V}_{2} {Z}_{2}}{{Z}_{2} + \frac{1}{j ω {C}_{2}}} = {V}_{1} - {V}_{2} - \frac{{V}_{1} {Z}_{1}}{{Z}_{1} + \frac{1}{j ω {C}_{1}}} + \frac{{V}_{2} {Z}_{2}}{{Z}_{2} + \frac{1}{ j ω {C}_{2}}} \qquad ( eq . 3 )$$

In Equation 3, V₁−V₂ represents the desired voltage across the unknown bio-impedance 102, and

$$- \frac{{V}_{1} {Z}_{1}}{{Z}_{1} + \frac{1}{j ω {C}_{1}}} + \frac{{V}_{2} {Z}_{2}}{{Z}_{2} + \frac{1}{ j ω {C}_{2}}}$$

represents the error term due to the input (parasitic) capacitances. For example, V₁−V₂ may represent a voltage drop caused by the bio-impedance 102 of the body of the subject.

$$- \frac{{V}_{1} {Z}_{1}}{{Z}_{1} + \frac{1}{j ω {C}_{1}}} + \frac{{V}_{2} {Z}_{2}}{{Z}_{2} + \frac{1}{j ω {C}_{2}}}$$

may represent a voltage drop caused by the impedances of the S+ branch, the impedances of the S− branch, the capacitance at the positive input of the inAmp 120, and the capacitance at the negative input of the inAmp 120. The technical task is to correct the measurement in a way to minimize the error term so that the measurement is less sensitive to errors caused by the impedances on the S+ and S− branches (including the parasitic capacitances at the inputs of inAmp 120). For example, the measurement of the bio-impedance 102 performed by the inAmp 120 may be improved by minimizing and/or compensating for the voltage drop caused by the impedances of the S+ branch, the impedances of the S− branch, the capacitance at the positive input of the inAmp 120, and the capacitance at the negative input of the inAmp 120.

A first technique can expose how sensitive the measurement is to changes in impedance on each one of the S+ branch and S− branch, and may make a correction to the results of the measurement based on the impedance sensitivity. The impedance on each one of the S+ branch and the S− branch can be resistive and/or capacitive. Accordingly, the technique can expose resistance and/or capacitance sensitivity on the S+ branch and the S− branch, and apply a correction accordingly. When the correction is applied appropriately, the error term included in Equation 3 may be reduced.

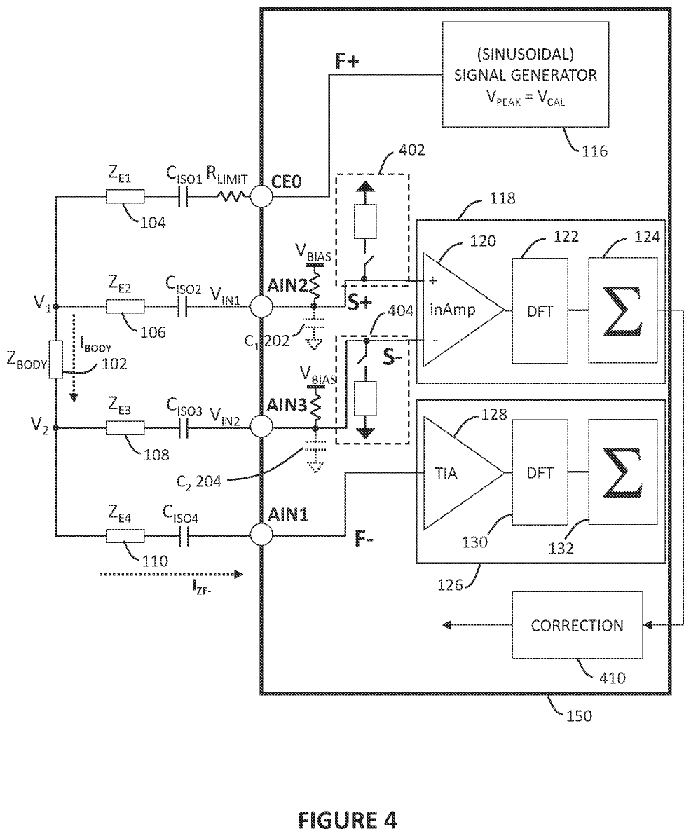

FIG. 4 illustrates circuitry which can perform a measurement that is less sensitive to errors caused by impedances (e.g., capacitance, and resistance) on the S+ branch and the S− branch, according to some embodiments of the disclosure. To expose the impedance sensitivity, circuitry 402 on the S+ branch can be implemented to add a pre-determined amount of impedance to the S+ branch, and circuitry 404 on the S− branch can be implemented to add a pre-determined amount of impedance on the S− branch. Circuitry 402 can be controlled to add a pre-determined amount of impedance (e.g., resistance or capacitance) to the S+ branch, e.g., at the positive input of inAmp 120. Circuitry 404 can be controlled to add a pre-determined amount of impedance (e.g., resistance or capacitance) to the S− branch, e.g., at the negative input of inAmp 120. For example, circuitry 402 can be controlled to couple a parallel capacitance C_{P} to the S+ branch, and circuitry 404 can be controlled to couple a parallel capacitance C_{P} to the S− branch. A capacitance value of the parallel capacitance C_{P} can be in the tens or hundreds pF.

By making some measurements while appropriately controlling the circuitry 402 and the circuitry 404, it is possible to expose the impedance sensitivity by observing the measurements made under different conditions. Specifically, three measurements under three different conditions can be made (in any suitable order):

1. Circuitry 402 and circuitry 404 do not add their respective pre-determined impedances. This measurement made by the inAmp 120 is referred to as V_{D1}.

2. Circuitry 402 can be controlled to add the pre-determined amount of impedance to the S+ branch. This measurement made by the inAmp 120 is referred to as V_{D2}. As an example, a small capacitance C_{P} is added by circuitry 402 as the pre-determined amount of impedance to the S+ branch.

3. Circuitry 404 can be controlled to add the pre-determined amount of impedance to the S− branch. This measurement made by the inAmp 120 is referred to as V_{D3}. As an example, a small capacitance C_{P} is added by circuitry 404 as the pre-determined amount of impedance to the S− branch.

Three measurements can be represented by the following:

$$\begin{matrix}{V}_{D 1} = {V}_{1} - {V}_{2} - \frac{{V}_{1} {Z}_{1}}{{Z}_{1} + \frac{1}{j ω {C}_{1}}} + \frac{{V}_{2} {Z}_{2}}{{Z}_{2} + \frac{1}{ j ω {C}_{2}}} & ( from eq . 3 ) \\ {V}_{D 2} = {V}_{1} - {V}_{2} - \frac{{V}_{1} {Z}_{1}}{{Z}_{1} + \frac{1}{j ω ( {C}_{1} + {C}_{P} )}} + \frac{{V}_{2} {Z}_{2}}{{Z}_{2} + \frac{1}{j ω {C}_{2}}} & ( eq . 4 ) \\ {V}_{D 3} = {V}_{1} - {V}_{2} - \frac{{V}_{1} {Z}_{1}}{{Z}_{1} + \frac{1}{j ω {C}_{1}}} + \frac{{V}_{2} {Z}_{2}}{{Z}_{2} + \frac{1}{j ω ( {C}_{2} + {C}_{P} )}} & ( eq . 5 )\end{matrix}$$

The derived impedance sensitivity, through adding the pre-determined impedance to the S+ branch and the S− branch and making various measurements under different conditions, can be used in correcting the impedance measurement, assuming that the error caused by impedance sensitivity scales with impedance, e.g., in a linear fashion. Correction circuitry 410 of FIG. 4 can process the measurements and perform correction accordingly. The correction performed by the correction circuitry 410 can involve finding differences in the measurements and scaling the differences appropriately. The scaling can be performed based on ratios of impedances on the branches. The scaled differences can be used to correct the measurement V_{D1}. In some cases, the differences can be combined with the measurement V_{D1} based on pre-determined scaling or weighing factors (e.g., determined based on a known model of how the sensitivity affects the measurement). By combining the measurement V_{D1} with weighted/scaled differences in the measurement caused by adding the pre-determined amount of impedance onto the branches, it is possible to take the sensitivity into account.

Two differences are computed, namely, V_{D2}−V_{D1} and V_{D3}−V_{D1}. The differences can expose the sensitivity of the measurement to a small pre-determined amount of impedance being added to the S+ branch and S− branch.

V_{D2} is subtracted by V_{D1}, which gives a difference in the measurements caused by adding the pre-determined amount of impedance on the S+ branch:

$${V}_{D 2} - {V}_{D 1} = \frac{{V}_{1} {Z}_{1}}{{Z}_{1} + \frac{1}{j ω {C}_{1}}} \cdot [ \frac{- \frac{{C}_{P}}{{C}_{1}} \cdot \frac{1}{j ω ( {C}_{1} + {C}_{P} )}}{{Z}_{1} + \frac{1}{j ω ( {C}_{1} + {C}_{P} )}} ] \qquad ( eq . 6 )$$

V_{D3} is subtracted by V_{D1}, which gives a difference in the measurements caused by adding the pre-determined amount of impedance on the S− branch:

$${V}_{D 3} - {V}_{D 1} = \frac{{V}_{2} {Z}_{2}}{{Z}_{2} + \frac{1}{j ω {C}_{2}}} \cdot [ \frac{\frac{{C}_{P}}{{C}_{2}} \cdot \frac{1}{j ω ( {C}_{2} + {C}_{P} )}}{{Z}_{2} + \frac{1}{j ω ( {C}_{2} + {C}_{P} )}} ] \qquad ( eq . 7 )$$

The result from Equation 6 can be scaled by

$$\frac{{C}_{1}}{{C}_{P}} ,$$

which is a ratio between the input (parasitic) capacitance at the positive input of inAmp 120 and the pre-determined amount of impedance, e.g., C_{P}, being added on the S+ branch.

The result from Equation 7 can be scaled by

$$\frac{{C}_{2}}{{C}_{P}} ,$$

which is a ratio between the input (parasitic) capacitance at the negative input of inAmp 120 and the pre-determined amount of impedance, e.g., C_{P}, being added on the S− branch.

The input (parasitic) capacitance C₁ on the S+ branch and the input (parasitic) capacitance C₂ on the S− branch can be measured. For instance, the input (parasitic) capacitances can be measured directly as a capacitance measurement. In another instance, the input (parasitic) capacitances can be measured indirectly using known resistors (e.g., a calibration resistor) instead of the unknown bio-impedance. The added pre-determined amount of impedance on the S+ branch and the S-branch, e.g., C_{P}, is also known.

To perform the correction and obtain a corrected measurement V_{DCorr}, the measurement V_{D1} is subtracted by the result in Equation 6 scaled by

$$\frac{{C}_{1}}{{C}_{P}}$$

and is also subtracted by the result in Equation 7 scaled by

$$\frac{{C}_{2}}{{C}_{P}} \text{:}$$

$${V}_{D Corr} = {V}_{D 1} - \frac{{C}_{1}}{{C}_{P}} ( {V}_{D 2} - {V}_{D 1} ) - \frac{{C}_{2}}{{C}_{P}} ( {V}_{D 3} - {V}_{D 1} ) \\ {V}_{D Corr} = {V}_{1} - {V}_{2} - \frac{{V}_{1} {Z}_{1}}{{Z}_{1} + \frac{1}{j ω {C}_{1}}} [ 1 - \frac{\frac{1}{j ω ( {C}_{1} + {C}_{P} )}}{{Z}_{1} + \frac{1}{j ω ( {C}_{1} + {C}_{P} )}} ] + \frac{{V}_{2}}{{Z}_{2} + \frac{1}{j ω {C}_{2}}} \cdot [ 1 - \frac{\frac{1}{j ω ( {C}_{2} + {C}_{P} )}}{{Z}_{2} + \frac{1}{j ω ( {C}_{2} + {C}_{P} )}} ] \qquad ( eq . 8 )$$

In Equation 9 (which is an expanded version of Equation 8), V₁−V₂ represents the the desired voltage across the unknown bio-impedance 102, and

$$- \frac{{V}_{1} {Z}_{1}}{{Z}_{1} + \frac{1}{j ω {C}_{1}}} [ 1 - \frac{\frac{1}{j ω ( {C}_{1} + {C}_{P} )}}{{Z}_{1} + \frac{1}{j ω ( {C}_{1} + {C}_{P} )}} ] + \frac{{V}_{2} {Z}_{2}}{{Z}_{2} + \frac{1}{j ω {C}_{2}}} \cdot [ 1 - \frac{\frac{1}{j ω ( {C}_{2} + {C}_{P} )}}{{Z}_{2} + \frac{1}{j ω ( {C}_{2} + {C}_{P} )}} ]$$

represents the error term.

Comparing the error terms in Equation 3 and Equation 9, it can be seen that the error terms are different. Note that if

$$\frac{1}{ω ( {C}_{1} + {C}_{P} )} ⪢ {Z}_{1}$$

and similarly,

$$\frac{1}{ω ( {C}_{2} + {C}_{P} )} ⪢ {Z}_{2} ,$$

then the terms

$$[ 1 - \frac{\frac{1}{j ω ( {C}_{1} + {C}_{P} )}}{{Z}_{1} + \frac{1}{j ω ( {C}_{1} + {C}_{P} )}} ] and [ 1 - \frac{\frac{1}{j ω ( {C}_{2} + {C}_{P} )}}{{Z}_{2} + \frac{1}{j ω ( {C}_{2} + {C}_{P} )}} ]$$

would be close to zero. When the terms

$$[ 1 - \frac{\frac{1}{j ω ( {C}_{1} + {C}_{P} )}}{{Z}_{1} + \frac{1}{j ω ( {C}_{1} + {C}_{P} )}} ] and [ 1 - \frac{\frac{1}{j ω ( {C}_{2} + {C}_{P} )}}{{Z}_{2} + \frac{1}{j ω ( {C}_{2} + {C}_{P} )}} ]$$

are close to zero, it means that the error term

$$- \frac{{V}_{1} {Z}_{1}}{{Z}_{1} + \frac{1}{j ω {C}_{1}}} [ 1 - \frac{\frac{1}{j ω ( {C}_{1} + {C}_{P} )}}{{Z}_{1} + \frac{1}{j ω ( {C}_{1} + {C}_{P} )}} ] + \frac{{V}_{2} {Z}_{2}}{{Z}_{2} + \frac{1}{j ω {C}_{2}}} \cdot [ 1 - \frac{\frac{1}{j ω ( {C}_{2} + {C}_{P} )}}{{Z}_{2} + \frac{1}{j ω ( {C}_{2} + {C}_{P} )}} ]$$

in Equation 9 would be close to zero as well. As a result, the error caused by the impedance sensitivity is effectively reduced or corrected out through the correction illustrated in Equation 8.

The correction scheme of Equations 8 and 9 has an unexpected technical advantage that the phase of the measurement is minimally affected. When

$$\frac{1}{ω ( {C}_{1} + {C}_{P} )} ⪢ {Z}_{1}$$

and similarly,

$$\frac{1}{ω ( {C}_{2} + {C}_{P} )} ⪢ {Z}_{2} ,$$

the error term of Equation 9 result in pure real numbers. In the absence of an imaginary error term, the desired voltage in Equation 9 remains intact and the phase is minimally affected by the correction scheme.

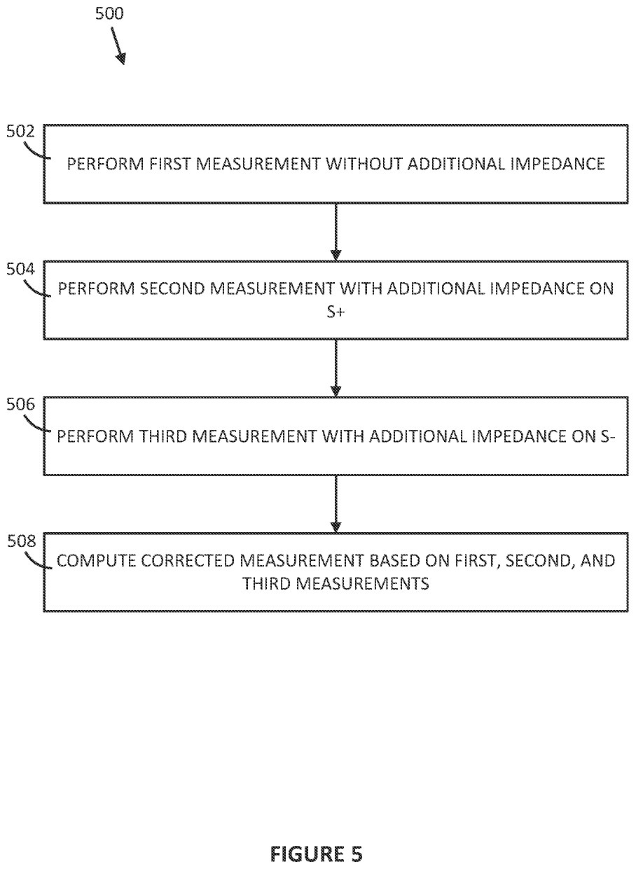

FIG. 5 is a flow diagram illustrating a method 500 for performing a measurement that is less sensitive to errors caused by impedances (e.g., capacitance, and resistance) on the S+ and S− branches, according to some embodiments of the disclosure. In 502, a first measurement, e.g., V_D1, is performed without any pre-determined amount of impedance added to the S+ and S− branches. In 504, a second measurement, e.g., V_D2, is performed with a pre-determined amount of impedance added to the S+ branch. In 506, a third measurement, e.g., V_D2,is performed with a pre-determined amount of impedance added to the S− branch. Measurements in 502, 504, and 506 can be performed in any suitable order. While the voltage measurements are made in 502, 504, and 506, current measurements on the F− branch can also be made. In 508, a corrected measurement V_DCorr can be computed by combining the first, second, and third measurements. For instance, a measurement to be corrected and differences in the measurements can be linearly combined. An example of a linear combination is illustrated by Equation 8 (e.g., by correction circuitry 410). In some cases, the corrected measurement V_DCorr can be used as part of the computation in a four-way impedance measurement scheme to derive the unknown bio-impedance 102. The resulting impedance measurement is more immune to errors caused by input (parasitic) capacitances on the S+ and S− branches, because the measurement's sensitivity to changes in impedance on the branches is taken into account.

Correction Based on Frequency Sensitivity

Second technique can expose how sensitive the measurement is to changes in frequency and can make a correction or compensation to the measurement based on the frequency sensitivity. Similar to the impedance sensitivity technique, multiple measurements may be made under different conditions, i.e., two different frequencies, and the measurements may be combined to form a corrected measurement. For example, the measurement to be corrected and a difference between the measurements made at two different frequencies can be linearly combined.

Bio-impedance may be predominately resistive, and should not change with respect to frequency. The second technique may assume that the error caused by frequency sensitivity linearly scales with frequency. The frequency sensitivity can be exposed by making measurements using at least a first frequency and a second frequency near the first frequency (the second frequency equals to the first frequency minus/plus a delta frequency), and the exposed frequency sensitivity (i.e., difference in measurements at the first frequency and the second frequency) can be applied in a correction scheme. When the correction is applied appropriately, the error term seen in Equation 9 can be eliminated or reduced.

Referring back to Equation 9, and substituting

$$\frac{1}{ω ( {C}_{1} + {C}_{P} )}$$

with k₁ and substituting

$$\frac{1}{ω ( {C}_{2} + {C}_{P} )}$$

with k₂, the error term in Equation 9 can be rewritten as:

$$Error term = - \frac{{V}_{1} {Z}_{1}}{{Z}_{1} - j {k}_{1}} [ \frac{j {Z}_{1}}{{k}_{1}} ] + \frac{{V}_{2} {Z}_{1}}{{Z}_{2} - j {k}_{2}} [ \frac{j {Z}_{2}}{{k}_{2}} ] ≅ \frac{{V}_{1} {Z}_{1}^{2}}{{k}_{1}^{2}} - \frac{{V}_{2} {Z}_{2}^{2}}{{k}_{2}^{2}} ≅ {V}_{1} {Z}_{1}^{2} {{ω}^{2} ( {C}_{1} + {C}_{P} )}^{2} - {V}_{2} {Z}_{2}^{2} {{ω}^{2} ( {C}_{2} + {C}_{p} )}^{2} \qquad ( eq . 10 )$$

The second technique can be applied to the corrected measurement of Equation 8. Based on the reformulation of the error term illustrated by Equation 10, the corrected measurement of Equation 8, measured at a first frequency, ω, can be given by:

V_{DCorr}(ω)=V₁−V₂+V₁Z₁²ω²(C₁+C_{P})²−V₂Z₂²ω²(C₂+C_{P})² (eq. 11)

A corrected measurement of Equation 8, measured at a second frequency near the first frequency ω+Δω, can be given by:

V_{DCorr}(ω+Δω)=V₁−V₂+V₁Z₁²(ω+Δω)²(C₁+C_{P})²−V₂Z₂²(ω+Δω)²(C₂+C_{P})² (eq. 12)

Subtracting the result of V_{DCorr}(ω+Δω) in Equation 12 by the result of V_{DCorr}(ω) of Equation 11, the difference in the two corrected measurements at two different frequencies can be given by:

ΔV_{Dcorr}=V₁Z₁²(C₁+C_{P})²2ωΔω−V₂Z₂²(C₂+C_{P})²2ωΔω (eq. 13)

The measurement to be corrected V_{Dcorr}(ω) and the difference in the two measurements ΔV_{DCorr} can be linearly combined to form a corrected measurement that takes the frequency sensitivity into account. For example, multiplying the result of ΔV_{DCorr} in Equation 13 by

$$\frac{ω}{2 Δ ω}$$

(e.g., a ratio of the first frequency and two times the delta frequency) and subtract from V_{Dcorr}(ω) of Equation 11 yields:

$${V}_{DCorr 2} = {V}_{DCorr} ( ω ) - Δ {V}_{DCorr} \cdot \frac{ω}{2 Δ ω} = {V}_{1} - {V}_{2} \qquad ( eq . 14 )$$

The error term previously seen in Equation 11 may be eliminated or reduced. Note that the correction technique based on frequency sensitivity may be valid as long as the measurements are made at the first frequency and second frequency near the first frequency. Using the first frequency and the delta frequency to form the ratio

$$\frac{ω}{2 Δ ω} ,$$

measurements obtained at the first frequency and the second frequency can be used to remove the error caused by frequency sensitivity. In other words, the second technique may be a point frequency correction for a given measurement made at a particular frequency point of interest, where the correction may be frequency specific. Delta frequency Δω can be selected based on the application.

In some cases, the second technique may be applied to an uncorrected measurement, such as V_{D1} of Equation 3.

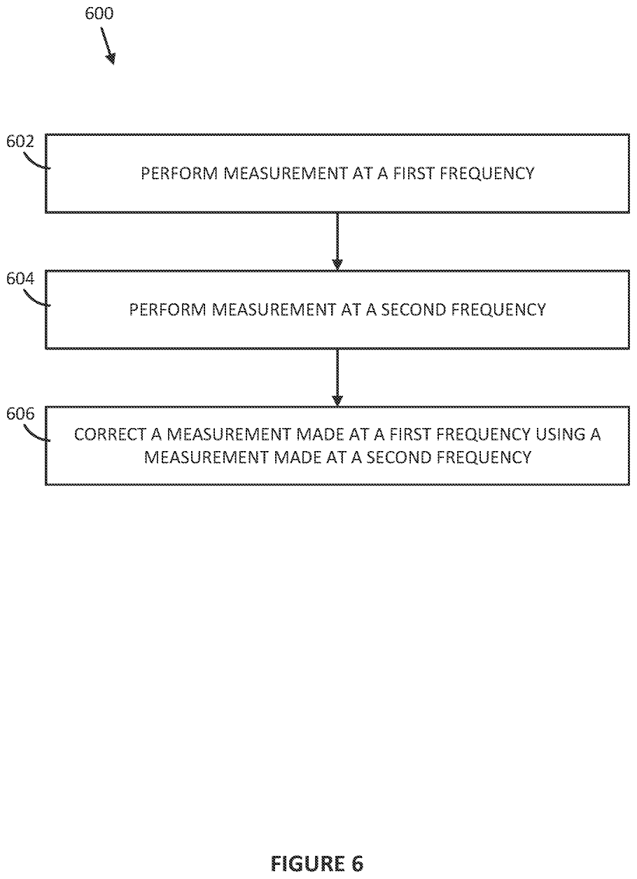

FIG. 6 is a flow diagram illustrating a method 600 for performing a measurement that is less sensitive to errors associated with frequency, according to some embodiments of the disclosure. In 602, a measurement may be made at a first frequency, e.g., in accordance with Equation 11. In 604, a measurement may be made at a second frequency near the first frequency, e.g., in accordance with Equation 12. In 606, correction can be applied, e.g., in accordance with Equation 14, to make the measurement more immune to non-idealities of the four-way measurement scheme, especially at high frequencies. The correction can be performed by correction circuitry 410 of FIG. 4. A corrected measurement V_{DCorr2} can be computed by combining the measurement to be corrected and a difference in the two measurements made at the first frequency and the second frequency. For instance, a measurement to be corrected and differences in the measurements can be linearly combined. In some cases, the corrected measurement V_{DCorr2} can be used as part of the computation in a four-way impedance measurement scheme to derive the unknown bio-impedance 102. The resulting impedance measurement may be more immune to errors due to frequency, because the measurement's sensitivity to changes in frequency on the branches is taken into account.

In some implementations, measurements are made at frequencies over a frequency range. Referring back to FIG. 4, the signal generator 116 can be configured to generate signals at different frequencies such that the measurements can be made at different frequencies. At least two measurements are made, e.g., one at the first frequency and one at the second frequency, to correct for frequency sensitivity of the measurement made at the first frequency. In some cases, multiple measurements at frequencies spanning over the frequency range with delta frequencies between the frequencies can be made. Preferably, for every measurement made a first frequency, a measurement is also made at the second frequency so that the correction can be performed. The measurements can be then used to correct for frequency sensitivity for a range of frequencies of interest. In other words, the sweep over the frequency range can provide frequency sensitivity information that can be used to perform the correction.

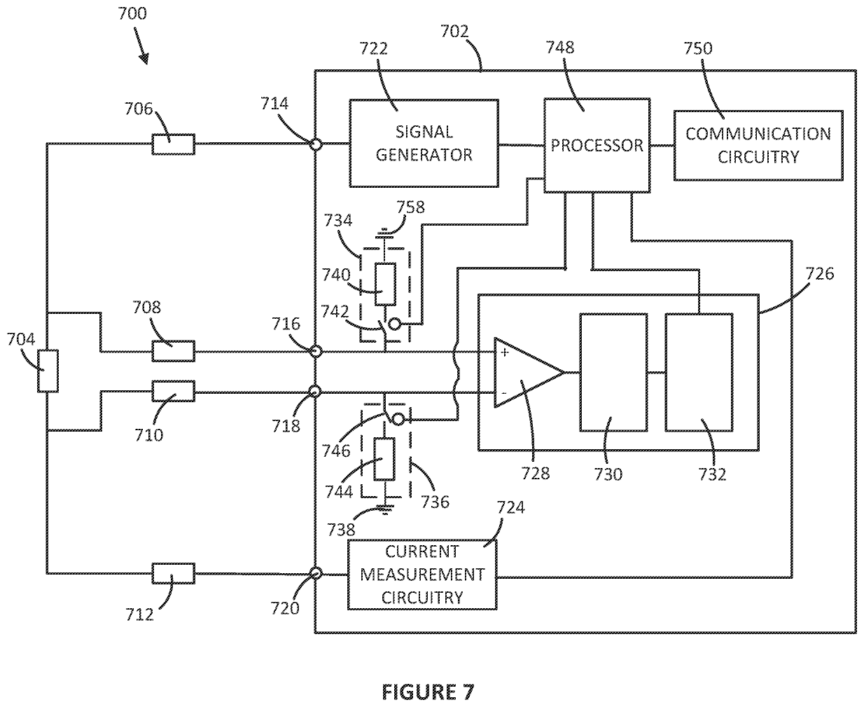

FIG. 7 illustrates an example measurement arrangement 700 that can implement the techniques described herein, according to some embodiments of the disclosure. In particular, the measurement arrangement 700 may include circuitry 702 that can implement one or both of the techniques in embodiments.

The measurement arrangement 700 may include a bio-impedance 704. The bio-impedance 704 may represent a portion of a body of a subject, where the portion of the body may present a certain impedance to current flow through the portion of the body. An amount of the bio-impedance 704 may be utilized for determining information about the subject, such as making determinations about the health of the subject.

The circuitry 702 may determine one or more electrical characteristics related to the bio-impedance 704 that can be utilized for determining the amount of the bio-impedance 704. In particular, the electrical characteristics may be utilized for determining an amount of impedance presented by the bio-impedance 704. For example, the circuitry 702 may apply a signal to the bio-impedance 704 and determine a voltage drop produced by the bio-impedance 704 due to the signal applied.

The circuitry 702 may be coupled to the bio-impedance 704 by a plurality of electrodes. For example, the circuitry 702 is coupled by a first electrode 706, a second electrode 708, a third electrode 710, and a fourth electrode 712. Each of the electrodes may comprise one or more of the features of the electrodes described throughout this disclosure. For example, each of the first electrode 706, the second electrode 708, the third electrode 710, and the fourth electrode 712 may include one or more of the features of the electrode 104 (FIG. 1), the electrode 106 (FIG. 1), the electrode 108 (FIG. 1), and the electrode 110 (FIG. 1). The electrodes may be positioned on the body of the subject, or some portion thereof, that corresponds to the bio-impedance 704. Each of the electrodes may present a contact impedance based on contact with a body of the subject. In particular, amounts of the contact impedances for the electrodes may be based on the quality of the contact between the electrode and the body of the subject. The first electrode 706 may present a first contact impedance (such as the contact impedance Z_{E1}), the second electrode 708 may present a second contact impedance (such as the contact impedance Z_{E2}), the third electrode 710 may present a third contact impedance (such as the contact impedance Z_{E3}), and the fourth electrode 712 may present a fourth contact impedance (such as the contact impedance Z_{E4}).

The circuitry 702 may include one or more pins that may be coupled to the electrodes. In some embodiments, the circuitry 702 may be included in an integrated circuit or chip, where the pins of the circuitry 702 may comprise contacts with the integrated circuit or chip, or may comprise electrical connectors of a system in which the circuitry 702 is implemented. In the illustrated embodiment, the circuitry 702 includes a first pin 714, a second pin 716, a third pin 718, and a fourth pin 720. The first pin 714 may be coupled to the first electrode 706, the second pin 716 may be coupled to the second electrode 708, the third pin 718 may be coupled to the third electrode 710, and the fourth pin 720 may be coupled to the fourth electrode 712. Wires, cables, or other means of coupling may be located between each of the pins and the electrode to which the pin is coupled. In some embodiments, isolation circuitry may be implemented between each of the electrodes and the corresponding pins. For example, capacitances (such as the capacitances C_{ISO1}, C_{ISO2}, C_{ISO3}, and C_{ISO4}) may be implemented between each of the electrodes and the corresponding pins. In other embodiments, the isolation circuitry may be omitted. Each of the corresponding electrodes, pins, and (where included) the capacitances may be referred to as a branch. For example, the first electrode 706 and the first pin 714 may be referred to as a first branch, the second electrode 708 and the second pin 716 may be referred to as a second branch, the third electrode 710 and the third pin 718 may be referred to as a third branch, and the fourth electrode 712 and the fourth pin 720 may be referred to as a fourth branch.

The circuitry 702 may include a signal generator 722. The signal generator 722 may include one or more of the features of the signal generator 116 (FIG. 1). The signal generator 722 may be coupled to a first end of the bio-impedance 704 via the first pin 714 and the first electrode 706. The signal generator 722 may produce a signal and apply the signal to the bio-impedance 704. The signal applied by the signal generator 722 to the bio-impedance 704 may cause current to flow across the bio-impedance 704 and a voltage drop to be produced across the bio-impedance 704. The signal applied to the bio-impedance 704 may comprise a signal having a frequency between 0 Hz and 999 million Hz in some embodiments. Further, the signal applied to the bio-impedance 704 may comprise a sinusoidal signal in some embodiments.

The circuitry 702 may further include current measurement circuitry 724. The current measurement circuitry 724 may include one or more of the features of the current measurement circuitry 126 (FIG. 1). The current measurement circuitry 724 may be coupled to a second end of the bio-impedance 704 via the fourth pin 720 and the fourth electrode 712, where the second end of the bio-impedance 704 is opposite from the first end of the bio-impedance 704. The current measurement circuitry 724 may determine an amount of current entering through the fourth pin 720 and generate an indication of the amount of current (such as a voltage that corresponds to the amount of current). Ideally, the current entering through the fourth pin 720 may be an entirety of the current being applied to the bio-impedance 704 via the signal generator 722. However, portions of the current being applied via the signal generator 722 may flow into other pins of the circuitry 702, as described throughout the disclosure.

The circuitry 702 may further include voltage measure circuitry 726. The voltage measurement circuitry 726 may include one or more of the features of the voltage measurement circuitry 118 (FIG. 1). For example, the voltage measurement circuitry 726 may include an inAmp 728, a DFT block 730, and a summation block 732. The voltage measurement circuitry 726 may determine an amount of the voltage drop across the bio-impedance 704. For example, the voltage measurement circuitry 726 may determine a voltage difference between the first end of the bio-impedance 704 and the second end of the bio-impedance 704.

The inAmp 728 of the voltage measurement circuitry 726 may be coupled to the bio-impedance 704 via the second pin 716, the second electrode 708, the third pin 718, and the third electrode 710. In particular, a first input of the inAmp 728 may be coupled to the first end of the bio-impedance 704 via the second pin 716 and the second electrode 708. A second input of the inAmp 728 may be coupled to the second end of the bio-impedance 704 via the third pin 718 and the third electrode 710. In some embodiments, the first input of the inAmp 728 may comprise a positive input of the inAmp 728 and the second input of the inAmp 728 may comprise a negative input of the inAmp 728. The inAmp 728 may determine a voltage difference between a voltage at the first end of the bio-impedance 704 and a voltage at the second end of the bio-impedance 704 and may output an indication of the voltage difference. Ideally, the inputs of the inAmp 728 will not allow current to flow into the inputs of the inAmp 728. However, the inputs may present high impedances in reality, where current may flow into the inputs of the inAmp 728. The impedances presented by the inputs may comprise parasitic capacitances, where the parasitic capacitances may be represented by grounded input impedances (such as the grounded input impedance 202 (FIG. 2) and the grounded input impedance 204 (FIG. 2)) coupled between the inputs of the inAmp 728 and a ground of the circuitry 702.

The circuitry 702 may further include first impedance circuitry 734 and second impedance circuitry 736. The first impedance circuitry 734 may include one or more of the features of the circuitry 402 (FIG. 4), and the second impedance circuitry 736 may include one or more of the features of the circuitry 404 (FIG. 4). The first impedance circuitry 734 may be coupled between the first end of the bio-impedance 704 (via the second pin 716 and the second electrode 708) and a ground 738 of the circuitry 702. The first impedance circuitry 734 may include a first impedance 740 and a first switch 742. The first impedance 740 may be coupled to the ground 738 and the first switch 742 may be coupled between the first impedance 740 and the first end of the bio-impedance 704. The first switch 742 may selectively couple the first impedance 740 to the first end of the bio-impedance 704. In particular, when the first switch 742 may couple the first impedance 740 to the first end of the bio-impedance 704 when closed and may decouple the first impedance 740 from the first end of the bio-impedance 704 when open. The first impedance 740 may comprise a first capacitance or a first resistance. The first switch 742 may comprise a mechanical switch or an electronic switch (such as a transistor configured in a switching arrangement).

The second impedance circuitry 736 may be coupled between the second end of the bio-impedance 704 (via the third pin 718 and the third electrode 710) and the ground 738 of the circuitry 702. The second impedance circuitry 736 may include a second impedance 744 and a second switch 746. The second impedance 744 may be coupled to the ground 738 and the second switch 746 may be coupled between the second impedance 744 and the second end of the bio-impedance 704. The second switch 746 may selectively couple the second impedance 744 to the second end of the bio-impedance 704. In particular, when the second switch 746 may couple the second impedance 744 to the second end of the bio-impedance 704 when closed and may decouple the second impedance 744 from the second end of the bio-impedance 704 when open. The second impedance 744 may comprise a second capacitance or a second resistance. The second switch 746 may comprise a mechanical switch or an electronic switch (such as a transistor configured in a switching arrangement). In some embodiments, the second capacitance or the second resistance may be the same as the first capacitance or the first resistance, where both the first switch 742 and the second switch 746 may be coupled to the first capacitance or the first resistance and selectively couple the first capacitance or the first resistance to the corresponding side of the bio-impedance 704. In other embodiments, the first impedance circuitry 734 and the second impedance circuitry 736 may be omitted.

The circuitry 702 may include a processor 748. The processor 748 may comprise one or more processors, one or more microprocessors, one or more microcomputers, or some combination thereof. The processor 748 may implement one or both of the techniques of correcting or compensating a determination of a value of the bio-impedance 704 described throughout. For example, the processor 748 may be coupled to the first switch 742 and the second switch 746 in some embodiments, where the processor 748 may implement the technique for correcting and/or compensating based on impedance sensitivity. The processor 748 may cause the first switch 742 to selectively couple the first impedance 740 to the first end of the bio-impedance 704 and the second switch 746 to selectively couple the second impedance 744 to the second end of the bio-impedance 704 to implement the correcting and/or compensating based on impedance sensitivity. In particular, the processor 748 may cause the first switch 742 to couple the first impedance 740 to the first end of the bio-impedance 704 and the second switch 746 to decouple the second impedance 744 from the second end of the bio-impedance 704.

The processor 748 may further be coupled to the voltage measurement circuitry 726 and cause the voltage measurement circuitry 726 to determine voltage differences between voltages at the first end of the bio-impedance 704 and voltages at the second end of the bio-impedance 704. A first voltage difference between a voltage at the first end of the bio-impedance 704 and a voltage at the second end of the bio-impedance 704 may be determined by the voltage measurement circuitry 726 with the circuitry 702 in the configuration with the first impedance 740 couple to the first end of the bio-impedance 704 and the second impedance 744 decoupled from the second end of the bio-impedance 704. The first voltage difference determined by the voltage measurement circuitry 726 may provide the voltage difference to be determined by equation 4. The processor 748 may further cause the first switch 742 to decouple the first impedance 740 from the first end of the bio-impedance 704 and the second switch 746 to couple the second impedance 744 to the second end of the bio-impedance 704. A second voltage difference between a voltage at the first end of the bio-impedance 704 and a voltage at the second end of the bio-impedance 704 may be determined by the voltage measurement circuitry 726 with the circuitry 702 in the configuration with the first impedance 740 decoupled from the first end of the bio-impedance 704 and the second impedance 744 coupled to the second end of the bio-impedance 704. The second voltage difference determined by the voltage measurement circuitry 118 may provide the voltage difference to be determined by equation 5. The processor 748 may further cause the first switch 742 to decouple the first impedance 740 from the first end of the bio-impedance 704 and the second switch 746 to decouple the second impedance 744 from the second end of the bio-impedance 704. A third voltage difference between a voltage at the first end of the bio-impedance 704 and a voltage at the second end of the bio-impedance 704 may be determined by the voltage measurement circuitry 726 with the circuitry 702 in the configuration with the first impedance 740 decoupled from the first end of the bio-impedance 704 and the second impedance 744 decoupled from the second end of the bio-impedance 704. A corrected voltage may be generated by correcting and/or compensating the third voltage difference with the first voltage difference and the second voltage difference in accordance with equation 8, where C₁ of equation 8 may be equal to the parasitic capacitance of the input of the inAmp 728 coupled to the first end of the bio-impedance 704, C_{P} of the second term of equation 8 may be equal to the first impedance 740, C₂ may be equal to the parasitic capacitance of the input of the inAmp 728 coupled to the second end of the bio-impedance 704, and C_{P} of the third term of equation 8 may be equal to the second impedance 744. Although an order of determining the voltage differences is described, it should be understood that the determinations of the voltage differences (including the selective coupling of the first impedance 740 and the second impedance 744) may be performed in any order.

The processor 748 may be coupled to the signal generator 722 in some embodiments, where the processor 748 may implement the technique for correcting and/or compensating based on frequency sensitivity. The processor 748 may cause the signal generator 722 to apply signals of different frequencies to the bio-impedance 704 to implement the correcting and/or compensating based on frequency sensitivity. In particular, the processor 748 may cause the signal generator 722 to apply a signal of a first frequency to the bio-impedance 704. A first voltage difference between a voltage at the first end of the bio-impedance 704 and a voltage at a second end of the bio-impedance 704 may be determined by the voltage measurement circuitry 726 with the configuration of the signal generator 722 applying the signal of the first frequency to the bio-impedance 704. The first voltage difference determined by the voltage measurement circuitry 726 may provide the first term of equation 14. The processor 748 may further cause the signal generator 722 to a apply a signal of a second frequency to the bio-impedance 704. A second voltage difference between a voltage at the first end of the bio-impedance 704 and a voltage at a second end of the bio-impedance 704 may be determined by the voltage measurement circuitry 726 with the configuration of the signal generator 722 applying the signal of the second frequency to the bio-impedance 704. The second voltage difference determined by the voltage measurement circuitry 726 may provide a portion of the first term of equation 14, where ω may be the first frequency applied by the signal generator and Aw may be an amount of difference between the first frequency applied by the signal generator 722 and the second frequency applied by the signal generator 722. A corrected voltage may be generated by correcting and/or compensating the first voltage difference with the the second voltage difference in accordance with equation 14.

In some embodiments where both the techniques are implemented, the technique of correcting and/or compensating based on impedance sensitivity may be performed prior to the technique for correcting and/or compensating based on frequency sensitivity. For example, the processor 748 may cause voltage differences to be determined with the first impedance and the second impedance selectively coupled in accordance with the technique for correcting and/or compensating based on impedance sensitivity while the signal generator 722 applies a signal of a first frequency. A first corrected voltage may be generated via the technique for correcting and/or compensating based on the impedance sensitivity utilizing the determined voltages at the first frequency. The processor 748 may cause voltage differences to be determined with the first impedance and the second impedance selectively coupled in accordance with the technique for correcting and/or compensating based on impedance sensitivity while the signal generator 722 applies a signal of a second frequency. A second corrected voltage may be generated via the technique for correcting and/or compensating based on the impedance sensitivity utilizing the determined voltages at the second frequency. The technique of correcting and/or compensating based on frequency sensitivity may then be applied to first corrected voltage and the second corrected voltage to generate a third corrected voltage.

The processor 748 may be coupled to the voltage measurement circuitry 726 and the current measurement circuitry 724. The processor 748 may receive the outputs of voltage measurement circuitry 726 and the current measurement circuitry 724. For example, the processor 748 may receive indications of the voltage differences determined by the voltage measurement circuitry 726. Further, the processor 748 may receive indications of the current determined by the current measurement circuitry 724. The processor 748 may further be coupled to communication circuitry 750 of the circuitry 702. The communication circuitry 750 may provide for wired communication and/or wireless communication with other elements. For example, the communication circuitry 750 may provide for wireless communication with a remote device. The processor 748 may utilize the communication circuitry 750 to provide the indications of the voltage differences and indications of the current to a remote device. In some embodiments, the circuitry 702 may include a memory device to store the indications of the voltage differences and the indications of the current prior to transmission of the indications to the remote device.

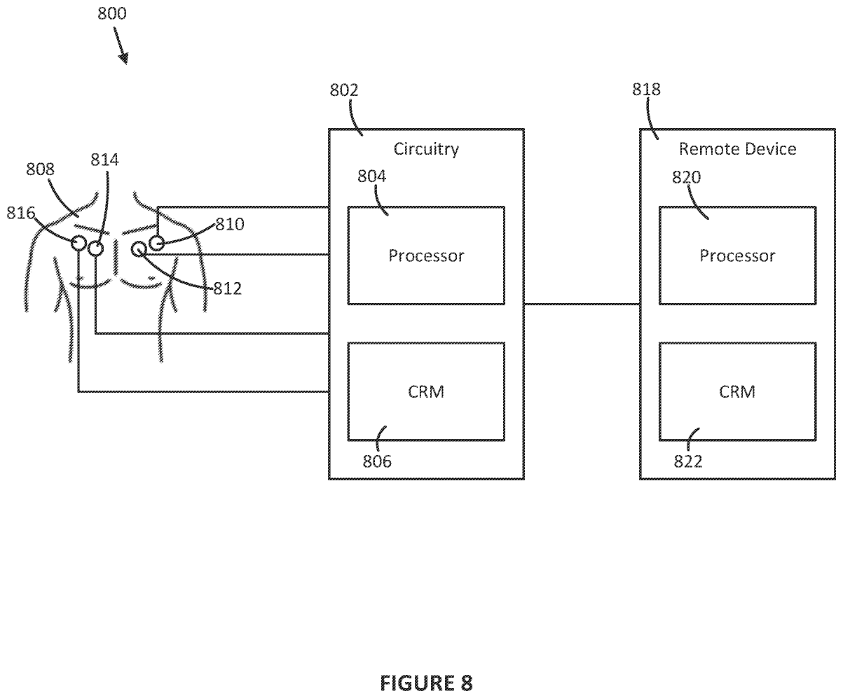

FIG. 8 illustrates an example system arrangement 800 that can implement the techniques described herein, according to some embodiments of the disclosure. In particular, the system arrangement 800 may implement one or both of the technique for correcting and/or compensating based on impedance sensitivity and the technique for correcting and/or compensating based on frequency sensitivity.

The system arrangement 800 may include circuitry 802. The circuitry 802 may include one or more of the features of the circuitry 150 (FIG. 1) and/or the circuitry 702 (FIG. 7). Further, the circuitry 802 may perform one or more of the procedures of the circuitry 150 and/or the circuitry 702. The circuitry 802 may include a processor 804, where the processor 804 includes one or more of the features of the processor 748 (FIG. 7). The circuitry 802 may further include one or more computer-readable media (CRM) 806. The CRM 806 may comprise non-transitory CRM in some embodiments. The CRM may have instructions stored thereon, wherein, when executed by the processor 804, cause the processor 804 to perform one or more of the procedures.

The circuitry 802 may be coupled to a body 808 of the subject by one or more electrodes. For example, the circuitry 802 is coupled to the body by a first electrode 810, a second electrode 812, a third electrode 814, and a fourth electrode 816 in the illustrated embodiment. Each of the electrodes may include one or more of the features of the electrodes described throughout this disclosure. For example, the first electrode 810 may include one or more of the features of the electrode 104 (FIG. 1) and/or the first electrode 706 (FIG. 7). The second electrode 812 may include one or more of the features of the electrode 106 (FIG. 1) and/or the second electrode 708 (FIG. 7). The third electrode 814 may include one or more of the features of the electrode 108 (FIG. 1) and/or the third electrode 710 (FIG. 7). The fourth electrode 816 may include one or more of the features of the electrode 110 (FIG. 1) and/or the fourth electrode 712 (FIG. 7).

The system arrangement 800 may further include a remote device 818. The remote device 818 may comprise a computer device, a server, or some combination thereof. The remote device 818 may be coupled (such as wiredly coupled or wirelessly coupled) to the circuitry 802. The remote device 818 may receive indications of voltage differences and/or indications of current from the circuitry 802. The indications of the voltage differences may indicate the voltage differences determined by the circuitry 802 between two of the electrodes in accordance with the procedures of determining voltage differences described throughout this disclosure, including the selective coupling of the impedances and/or the multiple frequencies of signals being applied to the body 808. The remote device 818 may perform the corrections and/or compensations in accordance the technique related to impedance sensitivity and/or the technique related to frequency sensitivity.

The remote device 818 may include a processor 820. The processor 820 may utilize the indications of the voltage differences to generate corrected voltages in accordance with the technique related to the impedance sensitivity and/or the technique related to frequency sensitivity. For example, the processor 820 may perform the calculations of one or more of the equations disclosed herein with the voltage differences to produce the corrected voltages. The remote device 818 may further include one or more CRM 822. The CRM 822 may comprise non-transitory CRM in some embodiments. The CRM 822 may have instructions stored thereon, wherein, when executed by the processor 820, cause the processor 820 to perform one or more of the procedures. In other embodiments, the remote device 818 may be implemented in the circuitry 802 and/or the procedures performed by the remote device 818 may be performed by the circuitry 802.

The circuitry 802 and the remote device 818 may perform the method 500 (FIG. 5) and/or the method 600 (FIG. 6). For example, in instances where the circuitry 802 and the remote device 818 perform the method 500, the circuitry 802 may perform a portion of the method 500 and the remote device 818 may perform another portion of the method. In some embodiments, the circuitry 802 may perform 502, 504, and 506, and the remote device 818 may perform 508. In instances where the circuitry 802 and the remote device 818 perform the method 600, the circuitry 802 may perform a portion of the method 600 and the remote device 818 may perform another portion of the method. In some embodiments, the circuitry 802 may perform 602 and 604, and the remote device 818 may perform 606. In embodiments where the circuitry 802 performs the procedures described being performed by the remote device 818, the circuitry 802 may perform the entirety of the method 500 and/or the method 600.

Additional Technical Advantages

Measuring bio-impedance can be particularly useful for measuring body impedance for detecting signs of congestive heart failure (e.g., by detecting fluid level of the lungs). Measuring bio-impedance can also be useful in electrical impedance tomography and spectroscopy to determine a composition of the body (e.g., imaging of tissues and bones) in a non-invasive manner by making bio-impedance measurements at different frequencies. Users such as athletes and patients can greatly benefit from such applications.

### EXAMPLE IMPLEMENTATIONS

The following examples are provided by way of illustration.

Example 1 may include circuitry for determining an amount of a bio-impedance of a portion of a body of a subject, the circuitry comprising first impedance circuitry coupled to a first pin of the circuitry, the first pin to be coupled to a first side of the portion of the body, wherein the first impedance circuitry is to selectively couple a first impedance to the first pin, second impedance circuitry coupled to a second pin of the circuitry, the second pin to be coupled to a second side of the portion of the body, wherein the second impedance circuitry is to selectively couple a second impedance to the second pin, and voltage measurement circuitry coupled to the first pin and the second pin, the voltage measurement circuitry to determine a first voltage difference between the first pin and the second pin with the first impedance coupled to the first pin and the second impedance decoupled from the second pin, and determine a second voltage difference between the first pin and the second pin with the first impedance decoupled from the first pin and the second impedance coupled to the second pin, the first voltage difference and the second voltage difference to be utilized for compensation to determine the amount of the bio-impedance.

Example 2 may include the circuitry of example 1, wherein the first impedance circuitry includes the first impedance that is coupled to a ground of the circuitry, and a first switch coupled between the first impedance and the first pin, the first switch to selectively couple the first impedance to the first pin, and the second impedance circuitry includes the second impedance that is coupled to the ground of the circuitry, and a second switch coupled between the second impedance and the second pin, the second switch to selectively couple the second impedance to the second pin.

Example 3 may include the circuitry of example 1, further comprising a signal generator to be coupled via a third pin to the body, the signal generator to apply a signal to the body for determination of the first voltage difference and the second voltage difference.

Example 4 may include the circuitry of example 3, wherein the signal applied to the body via the signal generator comprises a sinusoidal signal.

Example 5 may include the circuitry of example 1, wherein the circuitry further comprises a processor coupled to the first impedance circuitry, the second impedance circuitry, and the voltage measurement circuitry, the processor to cause the first impedance circuitry to couple the first impedance to the first pin, cause the voltage measurement circuitry to determine the first voltage difference while the first impedance circuitry has the first impedance coupled to the first pin, cause the second impedance circuitry to couple the second impedance to the second pin, and cause the voltage measurement circuity to determine the second voltage difference while the second impedance circuitry has the second impedance coupled to the second pin.

Example 6 may include the circuitry of example 5, wherein the processor is further to cause the first impedance circuitry to decouple the first impedance from the first pin, cause the second impedance circuitry to decouple the second impedance from the second pin, and cause the voltage measurement circuitry to determine a third voltage difference while the first impedance circuitry has the first impedance decoupled from the first pin and the second impedance decoupled from the second pin, wherein the third voltage difference is to be compensated via the first voltage difference and the second voltage difference to determine the amount of the bio-impedance.

Example 7 may include the circuitry of example 1, wherein the first impedance comprises a first capacitor, and wherein the second impedance comprises a second capacitor.

Example 8 may include the circuitry of example 1, wherein the first pin to is be coupled to a first electrode, the first electrode to be positioned on a first end of the portion of the body of the subject, wherein the second pin is to be coupled to a second electrode, the second electrode to be positioned on a second end of the portion of the body of the subject, and wherein the portion of the body of the subject produces the bio-impedance.

Example 9 may include a system for determining a value of a bio-impedance of a portion of a body of a subject, comprising a first electrode to be positioned on a first end of the portion of the body, a second electrode to be positioned on a second end of the portion of the body, and circuitry coupled to the first electrode and the second electrode, the circuitry to determine voltage differences between the first electrode and the second electrode, the circuitry comprising first impedance circuitry coupled to the first electrode, the first impedance circuitry to selectively couple a first impedance between the first electrode and a ground of the circuitry, second impedance circuitry coupled to the second electrode, the second impedance circuitry to selectively couple a second impedance between the second electrode and the ground of the circuitry, and voltage measurement circuitry coupled to the first electrode and the second electrode, the voltage measurement circuitry to determine the voltage differences between the first electrode and the second electrode with selective coupling of the first impedance between the first electrode and the ground of the circuitry and selective coupling of the second impedance between the second electrode and the ground of the circuitry.

Example 10 may include the system of example 9, wherein to determine the voltage differences between the first electrode and the second electrode with selective coupling of the first impedance and selective coupling of the second impedance includes to determine a first voltage difference between the first electrode and the second electrode with the first impedance coupled between the first electrode and the ground of the circuitry and the second impedance decoupled from between the second electrode and the ground of the circuitry, and determine a second voltage difference between the first electrode and the second electrode with the first impedance decoupled from between the first electrode and the ground of the circuitry and the second impedance coupled between the second electrode and the ground of the circuitry, the first voltage difference and the second voltage difference utilized for compensation of a third voltage difference to determine the value of the bio-impedance.

Example 11 may include the system of example 10, wherein the third voltage difference is determined with the first impedance decoupled from between the first electrode and the ground of the circuitry and the second impedance decoupled from between the second electrode and the ground of the circuitry.

Example 12 may include the system of example 9, wherein the first impedance circuitry includes the first impedance that is coupled to the ground of the circuitry, and a first switch coupled between the first impedance and the first electrode, the first switch to selectively couple the first impedance to the first electrode, and the second impedance circuitry includes the second impedance that is coupled to the ground of the circuitry, and a second switch coupled between the second impedance and the second electrode, the second switch to selectively couple the second impedance to the second electrode.

Example 13 may include the system of example 12, wherein the circuitry further comprises a controller coupled to the first switch and the second switch, wherein the controller causes the first switch and the second switch to transition states to selectively couple the first impedance to the first electrode and the second impedance to the second electrode.

Example 14 may include the system of example 9, wherein the circuitry includes an instrumentation amplifier (inAmp) with a positive input of the inAmp coupled to the first electrode and a negative input of the inAmp coupled to the second electrode, the inAmp utilized to determine the voltage differences between the first electrode and the second electrode.

Example 15 may include the system of example 9, wherein the circuitry further comprises a signal generator coupled to a third electrode, the third electrode to be positioned on the body, wherein the signal generator is to apply signals to the body to produce the voltage differences.

Example 16 may include the system of example 9, wherein the first impedance comprises a first capacitor, and wherein the second impedance comprises a second capacitor.

Example 17 may include a process for determining a value of a bio-impedance of a portion of a body of a subject, comprising determining, by circuitry, a first voltage difference between a first electrode positioned at a first end of the portion of the body and a second electrode positioned at a second end of the portion of the body with the circuitry having a first configuration, changing, by the circuitry, from the first configuration to a second configuration after the first voltage difference is determined, and determining, by the circuitry, a second voltage difference between the first electrode and the second electrode with the circuitry having the second configuration, the first voltage difference and the second voltage difference to be utilized for compensation to determine the value of the bio-impedance.

Example 18 may include the process of example 17, wherein the first configuration has a first impedance of the circuitry coupled to the first electrode and a second impedance of the circuitry decoupled from the second electrode, wherein the second configuration has the first impedance decoupled from the first electrode and the second impedance coupled to the second electrode, and wherein changing from the first configuration to the second configuration comprises decoupling, by the circuitry, the first impedance from the first electrode, and coupling, by the circuitry, the second impedance to the second electrode.

Example 19 may include the process of example 17, wherein determining the first voltage difference between the first electrode and the second electrode includes comparing, by a voltage measurement circuitry of the circuitry, a first voltage of the first electrode and a first voltage of the second electrode with the circuitry having the first configuration, and outputting, by the voltage measurement circuitry, the first voltage difference based on the comparing of the first voltage of the first electrode and the first voltage of the second electrode, and determining the second voltage difference between the first electrode and the second electrode includes comparing, by the voltage measurement circuitry, a second voltage of the first electrode and a second voltage of the second electrode with the circuitry having the second configuration, and outputting, by the voltage measurement circuitry, the second voltage difference based on the comparing of the second voltage of the second electrode and the second voltage of the second electrode.

Example 20 may include the process of example 17, further comprising applying, by a signal generator of the circuitry, a signal to the body to produce the first voltage difference and the second voltage difference.

Example 21 may include circuitry for determining an amount of a bio-impedance of a portion of a body of a subject, the circuitry comprising a signal generator to apply signals to the portion of the body, the signal generator to apply a first signal with a first frequency to the portion of the body and apply a second signal with a second frequency to the portion of the body, and voltage measurement circuitry coupled to a first pin that is coupled to a first side of the portion of the body and a second pin that is coupled to a second side of the portion of the body, the voltage measurement circuitry to determine a first voltage difference between the first pin and the second pin with the first signal with the first frequency applied to the portion of the body, and determine a second voltage difference between the first pin and the second pin with the second signal with the second frequency applied to the portion of the body, the first voltage difference and the second voltage difference to be utilized for compensation to determine the amount of the bio-impedance.

Example 22 may include circuitry for determining an amount of a bio-impedance of a portion of a body, the circuitry comprising a signal generator to apply a first signal with a first frequency to the portion of the body and apply a second signal with a second frequency to the portion of the body, first impedance circuitry coupled to a first pin of the circuitry, the first pin coupled to a first side of the portion of the body, wherein the first impedance circuitry is to selectively couple a first impedance to the first pin, second impedance circuitry coupled to a second pin of the circuitry, the second pin to be coupled to a second side of the portion of the body, wherein the second impedance circuitry is to selectively couple a second impedance to the second pin, and voltage measurement circuitry coupled to the first pin and the second pin, the voltage measurement circuitry to determine a first set of voltage differences with the first signal with the first frequency applied to the portion of the body, the first impedance circuitry selectively coupling the first impedance to the first pin, and the second impedance circuitry selectively coupling the second impedance to the second pin, and the voltage measurement circuitry to determine a second set of voltage differences with the second signal with the second frequency applied to the portion of the body, the first impedance circuitry selectively coupling the first impedance to the first pin, and the second impedance circuitry selectively coupling the second impedance to the second pin, the first set of voltage differences and the second set of voltage differences to be utilized for errors due to electrode contact impedance to determine the amount of the bio-impedance.

The foregoing outlines features of one or more embodiments of the subject matter disclosed herein. These embodiments are provided to enable a person having ordinary skill in the art (PHOSITA) to better understand various aspects of the present disclosure. Certain well-understood terms, as well as underlying technologies and/or standards may be referenced without being described in detail. It is anticipated that the PHOSITA will possess or have access to background knowledge or information in those technologies and standards sufficient to practice the teachings of the present disclosure.

The PHOSITA will appreciate that they may readily use the present disclosure as a basis for designing or modifying other processes, structures, or variations for carrying out the same purposes and/or achieving the same advantages of the embodiments introduced herein. The PHOSITA will also recognize that such equivalent constructions do not depart from the spirit and scope of the present disclosure, and that they may make various changes, substitutions, and alterations herein without departing from the spirit and scope of the present disclosure.

Note that the activities discussed above with reference to the FIGURES are applicable to any integrated circuit that involves signal processing (for example, gesture signal processing, video signal processing, audio signal processing, analog-to-digital conversion, digital-to-analog conversion), particularly those that can execute specialized software programs or algorithms, some of which may be associated with processing digitized real-time data. Certain embodiments can relate to multi-DSP, multi-ASIC, or multi-SoC signal processing, floating point processing, signal/control processing, fixed-function processing, microcontroller applications, etc. In certain contexts, the features discussed herein can be applicable to medical systems, scientific instrumentation, wireless and wired communications, radar, industrial process control, audio and video equipment, current sensing, instrumentation (which can be highly precise), and other digital-processing-based systems. Moreover, certain embodiments discussed above can be provisioned in digital signal processing technologies for medical imaging, patient monitoring, medical instrumentation, and home healthcare. This could include, for example, pulmonary monitors, accelerometers, heart rate monitors, or pacemakers, along with peripherals therefor. Other applications can involve automotive technologies for safety systems (e.g., stability control systems, driver assistance systems, braking systems, infotainment and interior applications of any kind). Furthermore, powertrain systems (for example, in hybrid and electric vehicles) can use high-precision data conversion, rendering, and display products in battery monitoring, control systems, reporting controls, maintenance activities, and others. In yet other example scenarios, the teachings of the present disclosure can be applicable in the industrial markets that include process control systems that help drive productivity, energy efficiency, and reliability. In consumer applications, the teachings of the signal processing circuits discussed above can be used for image processing, auto focus, and image stabilization (e.g., for digital still cameras, camcorders, etc.). Other consumer applications can include audio and video processors for home theater systems, DVD recorders, and high-definition televisions. Yet other consumer applications can involve advanced touch screen controllers (e.g., for any type of portable media device). Hence, such technologies could readily part of smartphones, tablets, security systems, PCs, gaming technologies, virtual reality, simulation training, etc.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

The particular embodiments of the present disclosure may readily include a system on chip (SoC) central processing unit (CPU) package. An SoC represents an integrated circuit (IC) that integrates components of a computer or other electronic system into a single chip. It may contain digital, analog, mixed-signal, and radio frequency functions: all of which may be provided on a single chip substrate. Other embodiments may include a multi-chip-module (MCM), with a plurality of chips located within a single electronic package and configured to interact closely with each other through the electronic package. Any module, function, or block element of an ASIC or SoC can be provided, where appropriate, in a reusable “black box” intellectual property (IP) block, which can be distributed separately without disclosing the logical details of the IP block. In various other embodiments, the digital signal processing functionalities may be implemented in one or more silicon cores in application-specific integrated circuits (ASICs), field-programmable gate arrays (FPGAs), and other semiconductor chips.

In some cases, the teachings of the present disclosure may be encoded into one or more tangible, non-transitory computer-readable mediums having stored thereon executable instructions that, when executed, instruct a programmable device (such as a processor or DSP) to perform the methods or functions disclosed herein. In cases where the teachings herein are embodied at least partly in a hardware device (such as an ASIC, IP block, or SoC), a non-transitory medium could include a hardware device hardware-programmed with logic to perform the methods or functions disclosed herein. The teachings could also be practiced in the form of Register Transfer Level (RTL) or other hardware description language such as VHDL or Verilog, which can be used to program a fabrication process to produce the hardware elements disclosed.

In example implementations, at least some portions of the processing activities outlined herein may also be implemented in software. In some embodiments, one or more of these features may be implemented in hardware provided external to the elements of the disclosed figures, or consolidated in any appropriate manner to achieve the intended functionality. The various components may include software (or reciprocating software) that can coordinate in order to achieve the operations as outlined herein. In still other embodiments, these elements may include any suitable algorithms, hardware, software, components, modules, interfaces, or objects that facilitate the operations thereof.

Additionally, some of the components associated with described microprocessors may be removed, or otherwise consolidated. In a general sense, the arrangements depicted in the figures may be more logical in their representations, whereas a physical architecture may include various permutations, combinations, and/or hybrids of these elements. It is imperative to note that countless possible design configurations can be used to achieve the operational objectives outlined herein. Accordingly, the associated infrastructure has a myriad of substitute arrangements, design choices, device possibilities, hardware configurations, software implementations, equipment options, etc.

Any suitably-configured processor component can execute any type of instructions associated with the data to achieve the operations detailed herein. Any processor disclosed herein could transform an element or an article (for example, data) from one state or thing to another state or thing. In another example, some activities outlined herein may be implemented with fixed logic or programmable logic (for example, software and/or computer instructions executed by a processor) and the elements identified herein could be some type of a programmable processor, programmable digital logic (for example, an FPGA, an erasable programmable read only memory (EPROM), an electrically erasable programmable read only memory (EEPROM)), an ASIC that includes digital logic, software, code, electronic instructions, flash memory, optical disks, CD-ROMs, DVD ROMs, magnetic or optical cards, other types of machine-readable mediums suitable for storing electronic instructions, or any suitable combination thereof. In operation, processors may store information in any suitable type of non-transitory storage medium (for example, random access memory (RAM), read only memory (ROM), FPGA, EPROM, electrically erasable programmable ROM (EEPROM), etc.), software, hardware, or in any other suitable component, device, element, or object where appropriate and based on particular needs. Further, the information being tracked, sent, received, or stored in a processor could be provided in any database, register, table, cache, queue, control list, or storage structure, based on particular needs and implementations, all of which could be referenced in any suitable timeframe. Any of the memory items discussed herein should be construed as being encompassed within the broad term ‘memory.’ Similarly, any of the potential processing elements, modules, and machines described herein should be construed as being encompassed within the broad term ‘microprocessor’ or ‘processor.’ Furthermore, in various embodiments, the processors, memories, network cards, buses, storage devices, related peripherals, and other hardware elements described herein may be realized by a processor, memory, and other related devices configured by software or firmware to emulate or virtualize the functions of those hardware elements.

Computer program logic implementing all or part of the functionality described herein is embodied in various forms, including, but in no way limited to, a source code form, a computer executable form, a hardware description form, and various intermediate forms (for example, mask works, or forms generated by an assembler, compiler, linker, or locator). In an example, source code includes a series of computer program instructions implemented in various programming languages, such as an object code, an assembly language, or a high-level language such as OpenCL, RTL, Verilog, VHDL, Fortran, C, C++, JAVA, or HTML for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

In the discussions of the embodiments above, the capacitors, buffers, graphics elements, interconnect boards, clocks, DDRs, camera sensors, converters, inductors, resistors, amplifiers, switches, digital core, transistors, and/or other components can readily be replaced, substituted, or otherwise modified in order to accommodate particular circuitry needs. Moreover, it should be noted that the use of complementary electronic devices, hardware, non-transitory software, etc. offer an equally viable option for implementing the teachings of the present disclosure.

In one example embodiment, any number of electrical circuits of the FIGURES may be implemented on a board of an associated electronic device. The board can be a general circuit board that can hold various components of the internal electronic system of the electronic device and, further, provide connectors for other peripherals. More specifically, the board can provide the electrical connections by which the other components of the system can communicate electrically. Any suitable processors (inclusive of digital signal processors, microprocessors, supporting chipsets, etc.), memory elements, etc. can be suitably coupled to the board based on particular configuration needs, processing demands, computer designs, etc. Other components such as external storage, additional sensors, controllers for audio/video display, and peripheral devices may be attached to the board as plug-in cards, via cables, or integrated into the board itself. In another example embodiment, the electrical circuits of the FIGURES may be implemented as standalone modules (e.g., a device with associated components and circuitry configured to perform a specific application or function) or implemented as plug-in modules into application-specific hardware of electronic devices.

Note that with the numerous examples provided herein, interaction may be described in terms of two, three, four, or more electrical components. However, this has been done for purposes of clarity and example only. It should be appreciated that the system can be consolidated in any suitable manner. Along similar design alternatives, any of the illustrated components, modules, and elements of the FIGURES may be combined in various possible configurations, all of which are clearly within the broad scope of this disclosure. In certain cases, it may be easier to describe one or more of the functionalities of a given set of flows by only referencing a limited number of electrical elements. It should be appreciated that the electrical circuits of the FIGURES and its teachings are readily scalable and can accommodate a large number of components, as well as more complicated/sophisticated arrangements and configurations. Accordingly, the examples provided should not limit the scope or inhibit the broad teachings of the electrical circuits as potentially applied to a myriad of other architectures.

Numerous other changes, substitutions, variations, alterations, and modifications may be ascertained to one skilled in the art and it is intended that the present disclosure encompass all such changes, substitutions, variations, alterations, and modifications as falling within the scope of the appended claims. In order to assist the United States Patent and Trademark Office (USPTO) and, additionally, any readers of any patent issued on this application in interpreting the claims appended hereto, Applicant wishes to note that the Applicant: (a) does not intend any of the appended claims to invoke 35 U.S.C. § 112(f) as it exists on the date of the filing hereof unless the words “means for” or “steps for” are specifically used in the particular claims; and (b) does not intend, by any statement in the disclosure, to limit this disclosure in any way that is not otherwise reflected in the appended claims.

What is claimed is:

1. Circuitry for determining an amount of a bio-impedance of a portion of a body of a subject, the circuitry comprising:
first impedance circuitry coupled to a first pin of the circuitry, the first pin to be coupled to a first side of the portion of the body, wherein the first impedance circuitry is to selectively couple a first impedance to the first pin;
second impedance circuitry coupled to a second pin of the circuitry, the second pin to be coupled to a second side of the portion of the body, wherein the second impedance circuitry is to selectively couple a second impedance to the second pin; and
voltage measurement circuitry coupled to the first pin and the second pin, the voltage measurement circuitry to:
determine a first voltage difference between the first pin and the second pin with the first impedance coupled to the first pin and the second impedance decoupled from the second pin; and
determine a second voltage difference between the first pin and the second pin with the first impedance decoupled from the first pin and the second impedance coupled to the second pin, the first voltage difference and the second voltage difference to be utilized for compensation for errors due to electrode contact impedance to determine the amount of the bio-impedance.

2. The circuitry of claim 1, wherein:
the first impedance circuitry includes:
the first impedance that is coupled to a ground of the circuitry; and
a first switch coupled between the first impedance and the first pin, the first switch to selectively couple the first impedance to the first pin; and
the second impedance circuitry includes:
the second impedance that is coupled to the ground of the circuitry; and
a second switch coupled between the second impedance and the second pin, the second switch to selectively couple the second impedance to the second pin.

3. The circuitry of claim 1 further comprising a signal generator to be coupled via a third pin to the body, the signal generator to apply a signal to the body for determination of the first voltage difference and the second voltage difference.

4. The circuitry of claim 3, wherein the signal applied to the body via the signal generator comprises a sinusoidal signal.

5. The circuitry of claim 1, wherein the circuitry further comprises a processor coupled to the first impedance circuitry, the second impedance circuitry, and the voltage measurement circuitry, the processor to:
cause the first impedance circuitry to couple the first impedance to the first pin;
cause the voltage measurement circuitry to determine the first voltage difference while the first impedance circuitry has the first impedance coupled to the first pin;
cause the second impedance circuitry to couple the second impedance to the second pin; and
cause the voltage measurement circuity to determine the second voltage difference while the second impedance circuitry has the second impedance coupled to the second pin.

6. The circuitry of claim 5, wherein the processor is further to:
cause the first impedance circuitry to decouple the first impedance from the first pin;
cause the second impedance circuitry to decouple the second impedance from the second pin; and
cause the voltage measurement circuitry to determine a third voltage difference while the first impedance circuitry has the first impedance decoupled from the first pin and the second impedance decoupled from the second pin, wherein the third voltage difference is to be compensated via the first voltage difference and the second voltage difference to determine the amount of the bio-impedance.

7. The circuitry of claim 1, wherein the first impedance comprises a first capacitor, and wherein the second impedance comprises a second capacitor.

8. The circuitry of claim 1, wherein the first pin to is be coupled to a first electrode, the first electrode to be positioned on a first end of the portion of the body of the subject, wherein the second pin is to be coupled to a second electrode, the second electrode to be positioned on a second end of the portion of the body of the subject, and wherein the portion of the body of the subject produces the bio-impedance.

9. A system for determining a value of a bio-impedance of a portion of a body of a subject, comprising:
a first electrode to be positioned on a first end of the portion of the body;
a second electrode to be positioned on a second end of the portion of the body; and
circuitry coupled to the first electrode and the second electrode, the circuitry to determine voltage differences between the first electrode and the second electrode, the circuitry comprising:
first impedance circuitry coupled to the first electrode, the first impedance circuitry to selectively couple a first impedance between the first electrode and a ground of the circuitry;
second impedance circuitry coupled to the second electrode, the second impedance circuitry to selectively couple a second impedance between the second electrode and the ground of the circuitry; and
voltage measurement circuitry coupled to the first electrode and the second electrode, the voltage measurement circuitry to determine the voltage differences between the first electrode and the second electrode with selective coupling of the first impedance between the first electrode and the ground of the circuitry and selective coupling of the second impedance between the second electrode and the ground of the circuitry.

10. The system of claim 9, wherein to determine the voltage differences between the first electrode and the second electrode with selective coupling of the first impedance and selective coupling of the second impedance includes to:
determine a first voltage difference between the first electrode and the second electrode with the first impedance coupled between the first electrode and the ground of the circuitry and the second impedance decoupled from between the second electrode and the ground of the circuitry; and
determine a second voltage difference between the first electrode and the second electrode with the first impedance decoupled from between the first electrode and the ground of the circuitry and the second impedance coupled between the second electrode and the ground of the circuitry, the first voltage difference and the second voltage difference utilized for compensation for errors due to electrode contact impedance of a third voltage difference to determine the value of the bio-impedance.

11. The system of claim 10, wherein the third voltage difference is determined with the first impedance decoupled from between the first electrode and the ground of the circuitry and the second impedance decoupled from between the second electrode and the ground of the circuitry.

12. The system of claim 9, wherein:
the first impedance circuitry includes:
the first impedance that is coupled to the ground of the circuitry; and
a first switch coupled between the first impedance and the first electrode, the first switch to selectively couple the first impedance to the first electrode; and
the second impedance circuitry includes:
the second impedance that is coupled to the ground of the circuitry; and
a second switch coupled between the second impedance and the second electrode, the second switch to selectively couple the second impedance to the second electrode.

13. The system of claim 12, wherein the circuitry further comprises a controller coupled to the first switch and the second switch, wherein the controller causes the first switch and the second switch to transition states to selectively couple the first impedance to the first electrode and the second impedance to the second electrode.

14. The system of claim 9 , wherein the circuitry includes an instrumentation amplifier (inAmp) with a positive input of the inAmp coupled to the first electrode and a negative input of the inAmp coupled to the second electrode, the inAmp utilized to determine the voltage differences between the first electrode and the second electrode.

15. The system of claim 9, wherein the circuitry further comprises a signal generator coupled to a third electrode, the third electrode to be positioned on the body, wherein the signal generator is to apply signals to the body to produce the voltage differences.

16. The system of claim 9, wherein the first impedance comprises a first capacitor, and wherein the second impedance comprises a second capacitor.

17. A process for determining a value of a bio-impedance of a portion of a body of a subject, comprising:
determining, by circuitry, a first voltage difference between a first electrode positioned at a first end of the portion of the body and a second electrode positioned at a second end of the portion of the body with the circuitry having a first configuration;
changing, by the circuitry, from the first configuration to a second configuration after the first voltage difference is determined; and
determining, by the circuitry, a second voltage difference between the first electrode and the second electrode with the circuitry having the second configuration, the first voltage difference and the second voltage difference to be utilized for compensation to determine the value of the bio-impedance.

18. The process of claim 17, wherein the first configuration has a first impedance of the circuitry coupled to the first electrode and a second impedance of the circuitry decoupled from the second electrode, wherein the second configuration has the first impedance decoupled from the first electrode and the second impedance coupled to the second electrode, and wherein changing from the first configuration to the second configuration comprises decoupling, by the circuitry, the first impedance from the first electrode, and coupling, by the circuitry, the second impedance to the second electrode.

19. The process of claim 17, wherein:
determining the first voltage difference between the first electrode and the second electrode includes:
comparing, by a voltage measurement circuitry of the circuitry, a first voltage of the first electrode and a first voltage of the second electrode with the circuitry having the first configuration; and
outputting, by the voltage measurement circuitry, the first voltage difference based on the comparing of the first voltage of the first electrode and the first voltage of the second electrode; and
determining the second voltage difference between the first electrode and the second electrode includes:
comparing, by the voltage measurement circuitry, a second voltage of the first electrode and a second voltage of the second electrode with the circuitry having the second configuration; and
outputting, by the voltage measurement circuitry, the second voltage difference based on the comparing of the second voltage of the second electrode and the second voltage of the second electrode.

20. The process of claim 17 further comprising applying, by a signal generator of the circuitry, a signal to the body to produce the first voltage difference and the second voltage difference.
